(19) United States
(12) Patent Application Publication
(10) Pub. No.: US 2012/0177660 A1
(43) Pub. Date: Jul. 12, 2012

(54) BIOMARKERS AND THERAPEUTIC TARGETS FOR TYPE 1 DIABETES

(75) Inventors: Jin-Xiong She, Martinez, GA (US); Sharad Purohit, Augusta, GA (US)

(73) Assignee: Georgia Health Sciences University Research Institute, Inc.

(21) Appl. No.: 13/345,067

(22) Filed: Jan. 6, 2012

Related U.S. Application Data
(60) Provisional application No. 61/430,348, filed on Jan. 6, 2011.

(51) Int. Cl. A61K 39/395; A61P 3/10; A61K 38/16; G01N 33/577; C40B 30/04

(52) U.S. Cl. 424/158.1; 436/501; 506/9; 514/6.9; 424/172.1

(57) ABSTRACT

Compositions and methods for determining a subject's risk of developing type 1 diabetes (T1D) and diabetic complications are provided. One embodiment provides a method involving measuring the levels of interleukin-1-receptor antagonist (IL-1ra) in a sample from the subject. In other embodiments, the method involves measuring the levels of MIP-1β, IL-8, MCP-1, MPO, SAA, IGFBP2, Adiponectin, or combinations thereof. Another embodiment provides preventing islet autoimmunity and T1D using agonist of IL-1ra, MIP-1β, IL-8, MCP-1, MPO, or a combination thereof. Another embodiment provides preventing islet autoimmunity, T1D and diabetic complications using antagonist of SAA, IGFBP2, Adiponectin, or a combination thereof.

### CROSS REFERENCE TO RELATED APPLICATIONS

This application claims benefit of and priority to U.S. Ser. No. 61/430,348 filed Jan. 6, 2011, the contents of which are incorporated herein by reference in its entirety.

### STATEMENT REGARDING FEDERALLY SPONSORED RESEARCH OR DEVELOPMENT

This invention was made with Government Support under Agreements 4R33HD050196, 4R33DK069878, 2RO1HD37800, 5R37DK032493, and P30DK057516 by the National Institutes of Health. The Government has certain rights in the invention.

### FIELD OF THE INVENTION

The invention is generally related to biomarkers and therapeutic targets for type 1 diabetes.

### BACKGROUND OF THE INVENTION

Type 1 diabetes (T1D) is an autoimmune disease that causes destruction of the insulin producing β-cells in the pancreatic islets as a result of the complex interactions between susceptibility genes and environmental triggers. A number of T1D susceptibility genes have been identified using genetic association studies (Todd, J. A. Nat Genet. 38, 731-33 (2006); Cooper, J. D. et al. Nat Genet. 40, 1399-1401 (2008)) and these genes, particularly the HLA-DR and HLA-DQ genes, can be used to identify subjects at increased risk for islet cell autoimmunity and diabetes. However, the susceptibility genes have poor specificity and sensitivity for disease prediction despite their widespread use in T1D screening studies (Nejentsev, S. et al. Diabet. Med. 16, 985-92 (1999); Rewers, M. et al. Diabetologia 39, 807-12 (1996); Carmichael, S. K. et al. Genet. Med. 5, 77-83 (2003); TEDDY Study Group. Pediatr. Diabetes 8, 286-98 (2007)). T1D prediction can be further improved by detecting autoantibodies against specific β-cell antigens (Schatz, D. et al. J. Clin. Invest 93, 2403-07 (1994); Ziegler, A. G. et al. Diabetes 38, 1320-25 (1989); Verge, C. F. et al. Diabetes 45, 926-33 (1996); Wenzlau, J. M. et al. Proceedings of the National Academy of Sciences 104, 17040-45 (2007)), which are the most specific and reliable T1D predictors that are now available.

However, better biomarkers are still urgently needed for multiple purposes. First, biomarkers that predate the onset of autoantibodies are critically needed for therapies aimed at inhibiting islet autoimmunity, which is believed to be the most efficient strategy for T1D prevention. Second, a subset of autoantibody-positive (AbP) subjects never progress to clinical disease despite the active autoimmune response. Elucidation of the underlying mechanisms should allow investigators to discover useful biomarkers for accurate disease prediction and provide valuable insight into disease pathogenesis and potentially novel therapeutic approaches.

It is an object of the invention to provide biomarkers to assist in assessing the propensity of a subject for developing diabetes.

It is an object of the invention to provide biomarkers to assess the outcome of therapies for type 1 diabetes.

Another object of the invention is to provide therapeutic targets for treating or inhibiting islet autoimmunity.

Still another object of the invention is to provide therapeutic targets for treating or inhibiting type 1 diabetes.

### SUMMARY OF THE INVENTION

Compositions and methods for determining a subject's risk of developing type 1 diabetes (T1D) are provided. One embodiment provides a method that involves measuring the levels of interleukin-1-receptor antagonist (IL-1ra) in a sample from the subject, wherein detection of low levels of IL-1ra in the sample compared to a control is an indication that the subject is at risk of developing T1D. For example, detection of less than about 1024 pg/ml IL-1ra in the sample can be an indication that the subject is at risk of developing T1D. Detection of decreasing IL-1ra levels over time can be further predictive of islet autoimmunity and T1D.

Another embodiment provides measuring the levels of monocyte inhibitory protein 1 beta (MIP-1β) in the sample, wherein detection of low levels of MIP-1β in the sample compared to a control is an indication that the subject is at risk of developing T1D.

Another embodiment provides measuring the levels of Interleukin 8 beta (IL-8) in the sample, wherein detection of low levels of IL-8 in the sample compared to a control is an indication that the subject is at risk of developing T1D.

Another embodiment provides measuring the levels of monocyte chemotactic protein 1 beta (MCP-1) in the sample, wherein detection of low levels of MCP-1 in the sample compared to a control is an indication that the subject is at risk of developing T1D.

Another embodiment provides measuring the levels of myeloperoxidase (MPO) in the sample, wherein detection of low levels of MPO in the sample compared to a control is an indication that the subject is at risk of developing T1D.

Another embodiment provides measuring the levels of serum amyloid A (SAA) in the sample, wherein detection of elevated levels of SAA in the sample compared to a control is an indication that the subject is at risk of developing T1D.

Another embodiment provides measuring the levels of serum insulin growth factor binding protein 2 (IGFBP2) in the sample, wherein detection of elevated levels of IGFBP2 in the sample compared to a control is an indication that the subject is at risk of developing T1D.

Another embodiment provides measuring the levels of serum adiponectin in the sample, wherein detection of elevated levels of adiponectin in the sample compared to a control is an indication that the subject is at risk of developing T1D.

In preferred embodiments, the sample from the subject is blood, plasma, or serum isolated from the subject. In certain embodiments, the method also involves assaying the sample for the presence of autoantibodies against islet antigens. In certain embodiments, the subject also has one or more known T1D susceptibility genes.

A method is also provided for treating type 1 diabetes in a subject, involving administering to the subject a composition having IL-1ra agonist. For example, in certain embodiments, the IL-1ra agonist is recombinant IL-1ra (anakinra, Kineret®).

A method is also provided for treating type 1 diabetes in a subject, involving administering to the subject a composition having IL-8 agonist. For example, in certain embodiments, the IL-8 agonist is recombinant IL-8.

A method is also provided for treating type 1 diabetes in a subject, involving administering to the subject a composition having MCP-1 agonist. For example, in certain embodiments, the MCP-1 agonist is recombinant MCP-1.

A method is also provided for treating type 1 diabetes in a subject, involving administering to the subject a composition having MPO agonist. For example, in certain embodiments, the MPO agonist is recombinant MPO.

A method is also provided for treating type 1 diabetes in a subject, involving administering to the subject a composition having SAA antagonist. For example, in certain embodiments, the SAA antagonist is a therapeutic antibody that specifically binds SAA.

A method is also provided for treating type 1 diabetes in a subject, involving administering to the subject a composition having IGFBP2 antagonist. For example, in certain embodiments, the IGFBP2 antagonist is a therapeutic antibody that specifically binds IGFBP2.

A method is also provided for treating type 1 diabetes in a subject, involving administering to the subject a composition having Adiponectin antagonist. For example, in certain embodiments, the Adiponectin antagonist is a therapeutic antibody that specifically binds Adiponectin.

### BRIEF DESCRIPTION OF THE DRAWINGS

FIGS. 1A and 1B are dot plots showing serum IL-1ra levels (ln(IL-1ra)) between T1D and AbN controls from general population (GP) and first degree relatives (FDR) in the DISCOVER dataset (FIG. 1A) the CONFIRM dataset (FIG. 1B). FIG. 1C is a graph showing the mean IL-1ra levels (ln(IL-1ra)) as a function of age (years), T1D status (T1D or AbN) and HLA DQB1 genotypes (201/201, 302/302, 201/302, 201/x, 302/x, x/x) in the CONFIRM dataset. This analysis examines the age interval of 10-60 years. A linear relationship was displayed here. x refers to all non-0201 and non-0302 DQB1 alleles. The bars to the left of each column of dots indicates the 5, 25, 50, 75, 90, 95, 99^{th} percentile distribution of the serum IL-1ra levels, from the bottom to the top respectively.

FIGS. 2A-2F are dot plots showing serum levels of MIP-1b (FIG. 2A), IL8 (FIG. 2B), MCP-1 (FIG. 2C), SAA (FIG. 2D), IGFBP2 (FIG. 2E), and Adiponectin (ADIPOQ) (FIG. 2F) in T1D subjects and AbN controls from the general population (GP) (n=700) and first degree relatives (FDR) of type 1 diabetes patients (n=700). Means for each group are shown and the dash lines indicate the 95^{th} percentile in T1D (FIG. 2A-2C) or all AbN (FIG. 2D-2F). P-values are between T1D and AbN groups. The bars to the left of each column of dots indicates the 5, 25, 50, 75, 90, 95, 99^{th} percentile distribution, starting from the bottom to the top, respectively, of the serum levels of MIP-1b, IL8, MCP-1, SAA, and IGFBP2.

FIG. 3A is a plot showing Log 2 serum MPO levels in AbN controls (left column) and T1D patients (right column). Each individual is represented by an open circle. The dashed line represents the 95^{th} percentile of MPO in T1D subjects. FIG. 3B is a box plot showing Log 2 means of serum MPO levels in AbN controls (left column) and T1D patients (right column). The box lines delineate the 25^{th}, 50^{th} and 75^{th} percentile respectively.

FIGS. 4A and 4B are plots showing Log 2 serum MPO levels in AbN general population (GP) control (FIG. 4A, left column), AbN first-degree relatives (FDR) (FIG. 4A, middle column), T1D patients (FIG. 4A, right column), Female AbN control (FIG. 4B, first column), Male AbN control (FIG. 4B, second column), Female T1D patients (FIG. 4B, third column), and Male T1D patients (FIG. 4B, fourth column). The dashed lines represent the 95^{th} percentile of MPO in T1D subjects.

FIG. 5A is a plot showing Log 2 serum SAA levels in AbN controls (left column) and T1D patients (right column). Each individual is represented by an open circle. The dashed line represents the 95^{th} percentile of SAA in T1D subjects. FIG. 5B is a box plot showing Log 2 means of serum SAA levels in AbN controls (left column) and T1D patients (right column). The box lines delineate the 25^{th}, 50^{th} and 75^{th} percentile respectively.

FIGS. 6A and 6B are plots showing Log 2 serum SAA levels in AbN general population (GP) control (FIG. 6A, left column), AbN first-degree relatives (FDR) (FIG. 6A, middle column), T1D patients (FIG. 9A, right column), Female AbN control (FIG. 6B, first column), Male AbN control (FIG. 6B, second column), Female T1D patients (FIG. 6B, third column), and Male T1D patients (FIG. 6B, fourth column). The dashed lines represent the 95th percentile of SAA in AbN subjects.

FIGS. 7A and 7B are regression trend lines according to age of the subjects. FIG. 7A shows the trend lines for T1D and AbN subjects by age. FIG. 7B shows the trend lines for male and female subjects by age.

FIGS. 8A and 8B are bar graphs showing mean SAA levels (log 2(SAA)) in AbN Controls (first bar), T1D patients without diabetic complications (middle bar), and T1D patients with photocoagulation (last bar) for males (FIG. 8B, first set of bars), females (FIG. 8B, second set of bars), or male and female subjects combined (FIG. 8A). FIG. 8C is a bar graph showing mean SAA levels (log 2(SAA)) in AbN Controls (first bar), T1D patients without diabetic complications (second bar), T1D patients with complication except nephropathy or amputation (third bar), T1D patients with nephropathy only (fourth bar), and T1D patients with nephropathy and other complications (fifth bar) for males (first set of bars) and females (second set of bars). FIG. 8D is a bar graph showing mean SAA levels (log 2(SAA)) in AbN Controls (first bar), T1D patients without diabetic complications (second bar), and T1D patients with nephropathy (third bar) for males (first set of bars) and females (second set of bars).

FIG. 9A-9E are bar graphs showing changes mean serum levels of serum proteins in nephropathy patients for IGFBP2 (FIG. 9A), IL-1Ra (FIG. 9B), IL-8 (FIG. 9C), MCP-1 (FIG. 9D) and MIP-1b (FIG. 9E) shows a bar chart for in AbN Controls (first bar), T1D patients without diabetic complications (second bar), T1D patients with other complication except nephropathy (third bar), T1D patients with nephropathy only (fourth bar), for females (first set of bars) and males (second set of bars).

FIG. 10A-10E are bar graphs showing changes mean serum levels of serum proteins in subjects, who suffered blindness, as a result of T1D for IGFBP2 (FIG. 10A), IL-1Ra (FIG. 10B), IL-8 (FIG. 10C), MCP-1 (FIG. 10D) and MIP-1b (FIG. 10E) shows a bar chart for in AbN Controls (first bar), T1D patients without diabetic complications (second bar), T1D patients with other complication except nephropathy (third bar), T1D patients with nephropathy only (fourth bar), for females (first set of bars) and males (second set of bars).

FIG. 11A-11C are bar graphs showing changes mean serum levels of serum proteins in subjects, who suffered cardio-vascular disease, as a result of T1D for IGFBP2 (FIG. 10A), IL-1Ra (FIG. 10B),) and MIP-1b (FIG. 10C) shows a bar chart for in AbN Controls (first bar), T1D patients without diabetic complications (second bar), T1D patients with other complication except nephropathy (third bar), T1D patients with nephropathy only (fourth bar), for females (first set of bars) and males (second set of bars).

FIG. 12A-12C are bar graphs showing changes mean serum levels of serum proteins in hypertensive T1D subjects for IGFBP2 (FIG. 12A), IL-8 (FIG. 12B),) and MIP-1b (FIG. 12C) shows a bar chart for in AbN Controls (first bar), T1D patients without diabetic complications (second bar), T1D patients with other complication except nephropathy (third bar), T1D patients with nephropathy only (fourth bar), for females (first set of bars) and males (second set of bars).

FIG. 13A-13B are bar graphs showing changes mean serum levels of serum proteins in T1D subjects, with degeneration of eye known as retinopathy for IL-8 (FIG. 13a),) and MIP-1b (FIG. 13B) shows a bar chart for in AbN Controls (first bar), T1D patients without diabetic complications (second bar), T1D patients with other complication except nephropathy (third bar), T1D patients with nephropathy only (fourth bar), for females (first set of bars) and males (second set of bars).

### DETAILED DESCRIPTION OF THE INVENTION

### I. Definitions

The term “sample” from a subject means a tissue (e.g., tissue biopsy), organ, cell (including a cell maintained in culture), cell lysate (or lysate fraction), or body fluid from a subject. Non-limiting examples of body fluids include blood, urine, plasma, serum, tears, lymph, bile, cerebrospinal fluid, interstitial fluid, aqueous or vitreous humor, colostrum, sputum, amniotic fluid, saliva, anal and vaginal secretions, perspiration, semen, transudate, exudate, and synovial fluid. In preferred embodiments, the sample of the disclosed methods is blood, plasma, or serum isolated from the subject.

The term “subject” means any individual who is the target of administration. The subject can be a vertebrate, for example, a mammal. Thus, the subject can be a human. The term does not denote a particular age or sex. A patient refers to a subject afflicted with a disease or disorder. The term “patient” includes human and veterinary subjects.

The term “therapeutically effective” means that the amount of the composition used is of sufficient quantity to ameliorate one or more causes or symptoms of a disease or disorder. Such amelioration only requires a reduction or alteration, not necessarily elimination.

By “treat” or “treatment” is meant the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained.

### II. Individual Serum Protein Biomarkers for Type 1 Diabetes

Compositions and methods are provided for determining a subject's risk of developing type 1 diabetes (T1D). The disclosed compositions include biomarkers that can be used to predict a subject's T1D diagnosis or prognosis. For example, the disclosed biomarkers can in some embodiments be used to determine whether the subject is at risk of developing islet autoimmunity. In other embodiments, the biomarkers can be used to determine whether the subject is at risk of developing T1D. For example, in some embodiments, the biomarkers can be used to determine whether the subject is at risk of progressing from islet autoimmunity to T1D.

Type 1 diabetes (T1D) is characterized by recurrent or persistent hyperglycemia, and is diagnosed by demonstrating any one of the following: fasting plasma glucose level ≧7.0 mmol/L (126 mg/dL); plasma glucose ≧11.1 mmol/L (200 mg/dL) two hours after a 75 g oral glucose load as in a glucose tolerance test; symptoms of hyperglycemia and casual plasma glucose ≧11.1 mmol/L (200 mg/dL); or glycated hemoglobin (Hb A1C) ≧6.5%.

Pre-diabetic states include people with fasting glucose levels from 100 to 125 mg/dL (5.6 to 6.9 mmol/L), who are considered to have impaired fasting glucose, and patients with plasma glucose at or above 140 mg/dL (7.8 mmol/L), but not over 200 mg/dL (11.1 mmol/L), two hours after a 75 g oral glucose load, who are considered to have impaired glucose tolerance.

In some embodiments, the compositions and method can determine that a subject has an at least 10%, 15%, 20%, 25%, 30%, 35%, 40%, 45%, at least 50% greater chance of developing islet autoimmunity compared to another subject having similar risk factors. In some embodiments, the compositions and method can determine that a subject has an at least 10%, 15%, 20%, 25%, 30%, 35%, 40%, 45%, at least 50% greater chance of developing T1D compared to another subject having similar risk factors. In some embodiments, the compositions and method can determine that a subject has an at least 10%, 15%, 20%, 25%, 30%, 35%, 40%, 45%, at least 50% greater chance of progressing from islet autoimmunity to T1D compared to another subject having similar risk factors.

The method can involve assaying for the levels of one or more biomarkers disclosed in a sample from the subject. In some embodiments, a decrease in biomarker levels in the sample compared to a control level is an indication that the subject is at risk of developing T1D. In other embodiments, an increase in biomarker levels in the sample compared to a control level is an indication that the subject is at risk of developing T1D.

Thus, the subject can in some embodiments be any human for which a diagnosis or prognosis relating to T1D is desired or warranted. In preferred embodiments of these methods, the sample is blood, plasma, or serum isolated from the subject.

Thus, in some embodiments, the subject has one or more T1D susceptibility genes. In some embodiments, the subject has one or more family members or relatives with T1D. In some embodiments, the subject has autoantibodies against islet antigens.

Other biomarkers have been identified that can be used alone or in combination with the one or more disclosed biomarkers. For example, additional biomarkers that are differentially expressed in type 1 diabetes can be used in the disclosed methods. Further, combinations of each and every disclosed biomarker is contemplated for use in the disclosed methods.

A. IL-1ra

Using a large discovery sample set, a larger confirmation sample set and a genetically screened high-risk cohort prospectively monitored for the development of islet cell autoimmunity and T1D, IL-1ra was identified and confirmed to be an excellent biomarker for both T1D and islet autoimmunity.

The disclosed biomarker can therefore be interleukin-1-receptor antagonist (IL-1ra). A method of determining a subject's risk of developing islet immunity and/or T1D is therefore disclosed. The method can in some embodiments involve measuring the levels of IL-1ra in a sample from the subject. This measurement can be done alone or in combination with any of the biomarkers disclosed herein.

In certain embodiments of this method, detection of less than about 100, 110, 120, 130, 140, 141, 142, 143, 144, 145, 156, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 300 pg/ml IL-1ra in the sample is an indication that the subject is at risk of developing T1D. Thus, in certain embodiments of this method, detection of less than about 1024 pg/ml IL-1ra in the sample is an indication that the subject is at risk of developing islet immunity and/or T1D. Thus, in certain embodiments of this method, levels of IL-1ra in the sample undetectable over background is an indication that the subject is at risk of developing islet immunity and/or T1D.

Levels of the biomarker can be compared to a control. For example, a negative control can be a sample from a healthy subject that does not have T1D. Likewise, a positive control can be a sample from a subject with T1D. Another suitable control can be a sample from a subject having islet autoantibodies but not T1D. The control can also be a reference value established based on data obtained from samples from a healthy subjects, subjects with T1D, subjects having islet autoantibodies, or a combination thereof.

Thus, in certain embodiments of this method, levels of IL-1ra in the sample lower than levels from a negative control is an indication that the subject is at risk of developing T1D. Likewise, levels of IL-1ra in the sample similar to or lower than levels from a positive control is an indication that the subject can be at risk of developing T1D.

In some embodiments, the method involves detecting changes in IL-1ra levels over time. For example, decreasing levels of IL-1ra in samples from the subject during the first 10 years of life can be an indication that the subject is at risk of developing islet immunity and/or T1D.

The disclosed methods can further involve assaying the sample for the presence of autoantibodies against islet antigens. Examples of islet autoantibody antigens include IAA, GADA and ICA512.

Thus, for example, detection in the sample of IL-1ra values lower than negative control values but not the presence of autoantibodies against islet antigens can be an indication that the subject is at risk of developing islet autoimmunity. Moreover, detection in the sample of autoantibodies against islet antigens and IL-1ra values lower than negative control can be an indication that the subject has islet autoimmunity and is at risk of developing T1D.

B. MIP-1β

The disclosed method can in some embodiments involve measuring the levels of monocyte inhibitory protein 1 beta (MIP-1β) in a sample from the subject. This measurement can be done alone or in combination with any of the biomarkers disclosed herein. In these embodiments, the detection of low levels of MIP-1β in the sample can be an indication that the subject is at risk of developing T1D.

In certain embodiments of this method, levels of MIP-1β in the sample lower than levels from a negative control is an indication that the subject is at risk of developing islet immunity and/or T1D. Likewise, levels of MIP-1β in the sample similar to or lower than levels from a positive control is an indication that the subject is at risk of developing islet immunity and/or T1D.

Thus, in certain embodiments of this method, levels of MIP-1β in the sample undetectable over background is an indication that the subject is at risk of developing islet immunity and/or T1D.

C. IL-8

The disclosed method can in some embodiments involve measuring the levels of Interleukin 8 (IL-8) in a sample from the subject. This measurement can be done alone or in combination with any of the biomarkers disclosed herein. In these embodiments, the detection of low levels of IL-8 in the sample can be an indication that the subject is at risk of developing T1D.

In certain embodiments of this method, levels of IL-8 in the sample lower than levels from a negative control is an indication that the subject is at risk of developing islet immunity and/or T1D. Likewise, levels of IL-8 in the sample similar to or lower than levels from a positive control is an indication that the subject is at risk of developing islet immunity and/or T1D.

Thus, in certain embodiments of this method, levels of IL-8 in the sample undetectable over background is an indication that the subject is at risk of developing islet immunity and/or T1D.

D. MCP-1

The disclosed method can in some embodiments involve measuring the levels of monocyte chemotactic protein 1 beta (MCP-1) in a sample from the subject. This measurement can be done alone or in combination with any of the biomarkers disclosed herein. In these embodiments, the detection of low levels of MCP-1 in the sample can be an indication that the subject is at risk of developing T1D.

In certain embodiments of this method, levels of MCP-1 in the sample lower than levels from a negative control is an indication that the subject is at risk of developing islet immunity and/or T1D. Likewise, levels of MCP-1 in the sample similar to or lower than levels from a positive control is an indication that the subject is at risk of developing islet immunity and/or T1D.

Thus, in certain embodiments of this method, levels of MCP-1 in the sample undetectable over background is an indication that the subject is at risk of developing islet immunity and/or T1D.

E. MPO

The disclosed method can in some embodiments involve measuring the levels of myeloperoxidase (MPO) in a sample from the subject. This measurement can be done alone or in combination with any of the biomarkers disclosed herein. In these embodiments, the detection of low levels of MPO in the sample can be an indication that the subject is at risk of developing T1D.

In certain embodiments of this method, levels of MPO in the sample lower than levels from a negative control is an indication that the subject is at risk of developing islet immunity and/or T1D. Likewise, levels of MPO in the sample similar to or lower than levels from a positive control is an indication that the subject is at risk of developing islet immunity and/or T1D.

In certain embodiments of this method, detection of less than about 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 420, 430, 440, 441, 442, 443, 444, 445, 456, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 600 pg/ml MCP-1 in the sample is an indication that the subject is at risk of developing islet immunity and/or T1D. Thus, in certain embodiments of this method, detection of less than about 128 pg/ml MCP-1 in the sample is an indication that the subject is at risk of developing islet immunity and/or T1D.

Thus, in certain embodiments of this method, levels of MPO in the sample undetectable over background is an indication that the subject is at risk of developing islet immunity and/or T1D.

F. IGFBP2

The disclosed method can in some embodiments involve measuring the levels of insulin growth factor binding protein 2 (IGFBP2) in a sample from the subject. This measurement can be done alone or in combination with any of the biomarkers disclosed herein. In these embodiments, the detection of increased levels of IGFBP2 in the sample can be an indication that the subject is at risk of developing T1D.

In certain embodiments of this method, levels of IGFBP2 in the sample higher than levels from a negative control is an indication that the subject is at risk of developing islet immunity and/or T1D. Likewise, levels of IGFBP2 in the sample similar to or higher than levels from a positive control is an indication that the subject is at risk of developing islet immunity and/or T1D.

G. Adiponectin

The disclosed method can in some embodiments involve measuring the levels of adiponectin in a sample from the subject. This measurement can be done alone or in combination with any of the biomarkers disclosed herein. In these embodiments, the detection of increased levels of adiponectin in the sample can be an indication that the subject is at risk of developing T1D.

In certain embodiments of this method, levels of adiponectin in the sample higher than levels from a negative control is an indication that the subject is at risk of developing islet immunity and/or T1D. Likewise, levels of adiponectin in the sample similar to or higher than levels from a positive control is an indication that the subject is at risk of developing islet immunity and/or T1D.

H. SAA

The disclosed method can in some embodiments involve measuring the levels of serum amyloid A (SAA) in a sample from the subject. This measurement can be done alone or in combination with any of the biomarkers disclosed herein. In these embodiments, the detection of increased levels of SAA in the sample can be an indication that the subject is at risk of developing T1D.

In certain embodiments of this method, levels of SAA in the sample higher than levels from a negative control is an indication that the subject is at risk of developing islet immunity and/or T1D. Likewise, levels of SAA in the sample similar to or higher than levels from a positive control is an indication that the subject is at risk of developing islet immunity and/or T1D.

In certain embodiments of this method, detection of greater than about 3600, 3650, 3700, 3750, 3800, 3850, 3900, 3950, 4000, 4050, 4100, 4150, 4200, 4250, 4300, 4350, 4400, 4450, 4500, 4550, 4600, 4650, 4700, 4750, 4800, 4850, 4900, 4950, 5000, 5050, 5100, 5150, 5200, 5250, 5300, 5350, 5400, 5450, 5500, 5550, 5600, 5650, 5700, 5750, 5800, 5850, 5900, 5950, 6000, 6050, 6100, 6150, 6200, 6250, 6300, 6350, 6400, 6450, or 6500 ng/ml SAA in the sample is an indication that the subject is at risk of developing islet immunity and/or T1D. Thus, in certain embodiments of this method, detection of greater than about 4,000 ng/ml SAA in the sample is an indication that the subject is at risk of developing islet immunity and/or T1D.

The disclosed method of determining a subject's risk of developing T1D can in some embodiments involve measuring the levels of SAA in a sample from the subject.

The disclosed method can further involve assaying the sample for the presence of autoantibodies against islet antigens. Thus, in certain embodiments of this method, detection in the sample of higher SAA but not the presence of autoantibodies against islet antigens is an indication that the subject is at risk of developing islet autoimmunity.

### III. Immunoassays

The disclosed methods can therefore involve measuring protein levels of IL-1ra, MIP-1β, IL-8, MCP-1, MPO, SAA, IGFBP2, Adiponectin, or a combination thereof, in a sample from the subject. In some aspects, the method is an immunoassay. Immunoassays, in their most simple and direct sense, are binding assays involving binding between antibodies and antigen. Thus, in some aspects, the method involves detecting IL-1ra, MIP-1β, IL-8, MCP-1, MPO, SAA, IGFBP2, Adiponectin, or a combination thereof, using one or more antibodies that specifically binds IL-1ra, MIP-1β, IL-8, MCP-1, MPO, SAA, IGFBP2, Adiponectin, or a combination thereof. The method can therefore involve detecting human IL-1ra, MIP-1β, IL-8, MCP-1, MPO, SAA, IGFBP2, Adiponectin, or a combination thereof. Antibodies that specifically bind human IL-1ra, MIP-1β, IL-8, MCP-1, MPO, SAA, IGFBP2 and Adiponectin are commercially available and can be produced using routine skill.

Therefore, measuring levels of the different biomarkers can include the detection of a biomarker:antibody complex. Measuring levels involves combining a specific antibody to the sample wherein a protein from the sample binds to the specific antibody and forms a biomarker:antibody complex. The biomarker:antibody complex can then be detected standard techniques in the art.

Many types and formats of immunoassays are known and all are suitable for detecting the disclosed biomarkers. Examples of immunoassays are enzyme linked immunosorbent assays (ELISAs), radioimmunoassays (RIA), radioimmune precipitation assays (RIPA), immunobead capture assays, Western blotting, dot blotting, gel-shift assays, Flow cytometry, protein arrays, multiplexed bead arrays, magnetic capture, in vivo imaging, fluorescence resonance energy transfer (FRET), and fluorescence recovery/localization after photobleaching (FRAP/FLAP).

For example, MPO levels can be measured using the CardioMPO™ kit (Cleveland Heart Lab), which is an enzyme immunoassay for the quantitative determination of MPO in human plasma. IL-1ra, MIP-1β, IL-8, MCP-1, MPO, SAA, IGFBP2 and Adiponectin can be assayed by Luminex kits from Millipore and BioRad.

In general, immunoassays involve contacting a sample suspected of containing a molecule of interest (such as the disclosed biomarkers) with an antibody to the molecule of interest or contacting an antibody to a molecule of interest (such as antibodies to the disclosed biomarkers) with a molecule that can be bound by the antibody, as the case may be, under conditions effective to allow the formation of immunocomplexes. Contacting a sample with the antibody to the molecule of interest or with the molecule that can be bound by an antibody to the molecule of interest under conditions effective and for a period of time sufficient to allow the formation of immune complexes (primary immune complexes) is generally a matter of simply bringing into contact the molecule or antibody and the sample and incubating the mixture for a period of time long enough for the antibodies to form immune complexes with, i.e., to bind to, any molecules (e.g., antigens) present to which the antibodies can bind. In many forms of immunoassay, the sample-antibody composition, such as a tissue section, ELISA plate, dot blot or Western blot, can then be washed to remove any non-specifically bound antibody species, allowing only those antibodies specifically bound within the primary immune complexes to be detected.

Immunoassays can include methods for detecting or quantifying the amount of a molecule of interest (such as the disclosed biomarkers or their antibodies) in a sample, which methods generally involve the detection or quantitation of any immune complexes formed during the binding process. In general, the detection of immunocomplex formation is well known in the art and can be achieved through the application of numerous approaches. These methods are generally based upon the detection of a label or marker, such as any radioactive, fluorescent, biological or enzymatic tags or any other known label.

A label can include a fluorescent dye, a member of a binding pair, such as biotin/streptavidin, a metal (e.g., gold), or an epitope tag that can specifically interact with a molecule that can be detected, such as by producing a colored substrate or fluorescence. Substances suitable for detectably labeling proteins include fluorescent dyes (also known as fluorochromes and fluorophores) and enzymes that react with colorometric substrates (e.g., horseradish peroxidase). The use of fluorescent dyes is generally preferred in the practice of the invention as they can be detected at very low amounts. Furthermore, in the case where multiple antigens are reacted with a single array, each antigen can be labeled with a distinct fluorescent compound for simultaneous detection. Labeled spots on the array are detected using a fluorimeter, the presence of a signal indicating an antigen bound to a specific antibody.

A modifier unit such as a radionuclide can be incorporated into or attached directly to any of the disclosed compositions by halogenation. In another aspect, the radionuclide can be attached to a linking group or bound by a chelating group, which is then attached to the compound directly or by means of a linker. Radiolabeling techniques such as these are routinely used in the radiopharmaceutical industry.

Labeling can be either direct or indirect. In direct labeling, the detecting antibody (the antibody for the molecule of interest) or detecting molecule (the molecule that can be bound by an antibody to the molecule of interest) include a label. Detection of the label indicates the presence of the detecting antibody or detecting molecule, which in turn indicates the presence of the molecule of interest or of an antibody to the molecule of interest, respectively. In indirect labeling, an additional molecule or moiety is brought into contact with, or generated at the site of, the immunocomplex. For example, a signal-generating molecule or moiety such as an enzyme can be attached to or associated with the detecting antibody or detecting molecule. The signal-generating molecule can then generate a detectable signal at the site of the immunocomplex. For example, an enzyme, when supplied with suitable substrate, can produce a visible or detectable product at the site of the immunocomplex. ELISAs use this type of indirect labeling.

As another example of indirect labeling, an additional molecule (which can be referred to as a binding agent) that can bind to either the molecule of interest or to the antibody (primary antibody) to the molecule of interest, such as a second antibody to the primary antibody, can be contacted with the immunocomplex. The additional molecule can have a label or signal-generating molecule or moiety. The additional molecule can be an antibody, which can thus be termed a secondary antibody. Binding of a secondary antibody to the primary antibody can form a so-called sandwich with the first (or primary) antibody and the molecule of interest. The immune complexes can be contacted with the labeled, secondary antibody under conditions effective and for a period of time sufficient to allow the formation of secondary immune complexes. The secondary immune complexes can then be generally washed to remove any non-specifically bound labeled secondary antibodies, and the remaining label in the secondary immune complexes can then be detected. The additional molecule can also be or include one of a pair of molecules or moieties that can bind to each other, such as the biotin/avadin pair. In this mode, the detecting antibody or detecting molecule should include the other member of the pair.

Other modes of indirect labeling include the detection of primary immune complexes by a two step approach. For example, a molecule (which can be referred to as a first binding agent), such as an antibody, that has binding affinity for the molecule of interest or corresponding antibody can be used to form secondary immune complexes, as described above. After washing, the secondary immune complexes can be contacted with another molecule (which can be referred to as a second binding agent) that has binding affinity for the first binding agent, again under conditions effective and for a period of time sufficient to allow the formation of immune complexes (thus forming tertiary immune complexes). The second binding agent can be linked to a detectable label or signal-generating molecule or moiety, allowing detection of the tertiary immune complexes thus formed. This system can provide for signal amplification.

Immunoassays that involve the detection of as substance, such as a protein or an antibody to a specific protein, include label-free assays, protein separation methods (i.e., electrophoresis), solid support capture assays, or in vivo detection. Label-free assays are generally diagnostic means of determining the presence or absence of a specific protein, or an antibody to a specific protein, in a sample. Protein separation methods are additionally useful for evaluating physical properties of the protein, such as size or net charge. Capture assays are generally more useful for quantitatively evaluating the concentration of a specific protein, or antibody to a specific protein, in a sample. Finally, in vivo detection is useful for evaluating the spatial expression patterns of the substance, i.e., where the substance can be found in a subject, tissue or cell.

### IV. Diagnostic Method

Diagnostic methods using the disclosed biomarkers detect T1D are further provided. The diagnostic method can involve detecting one or more of the disclosed biomarkers. For example, the diagnostic system can involve the use of an immunoassay to detect levels of biomarkers in a sample from a subject.

The disclosed method system can further involve the use of a computer system to compare levels of the one or more of the disclosed biomarkers to control values. For example, the computer system can use an algorithm to compare levels of two or more biomarkers and provide a score representing the risk of disease onset based on detected differences.

This algorithm can in some embodiments weigh multiple parameters. For example, in some embodiments, the algorithm gives weight depending on which biomarker demonstrates differences, e.g., more weight to differences in IL-1ra levels over other biomarkers. In some embodiments, the algorithm weighs the extent of elevation or decrease in biomarkers levels compared to the control. For example, a 50% reduction in biomarker levels may be weighted more than a 20% reduction in the same biomarker. In other embodiments, the algorithm gives weight to differences in biomarker levels for a combination of biomarkers.

Therefore, also provided is an apparatus for use in predicting the onset of islet immunity and/or T1D in a subject that includes an input means for entering biomarker level values from a sample of the subject, a processor means for comparing the values to control values, an algorithm for giving weight to specified parameters, and an output means for giving a score representing the risk of disease onset.

### V. Method of Treating T1D

Also disclosed is a method of treating or inhibit islet immunity in a subject that involves administering to the subject a composition having a therapeutically effective amount of an IL-1ra agonist, an MIP-1β agonist, an IL-8 agonist, an MCP-1 agonist, an MPO agonist, an SAA antagonist, an IGFBP2 antagonist, an Adiponectin antagonist, or a combination thereof.

A. Using Agonists

Thus, a method of treating or inhibiting islet immunity and/or T1D in a subject is disclosed that involves administering to the subject a composition having a therapeutically effective amount of IL-1ra agonist. In some embodiments, the method involves measuring the levels of IL-1ra in a sample from the subject. In these embodiments, the IL-1ra agonist can be administered to subjects if the sample from the subject has IL-1ra levels lower than control levels, e.g., less than about 200 pg/ml. In preferred embodiments of this method, the sample is blood, plasma, or serum isolated from the subject.

Also disclosed is a method of treating or inhibit islet immunity and/or T1D in a subject that involves administering to the subject a composition having a therapeutically effective amount of a MIP-1β agonist. In some embodiments, the method involves measuring the levels of MIP-1β in a sample from the subject. In these embodiments, the MIP-1β agonist can be administered to subjects if the sample from the subject has MIP-1β levels lower than control levels.

Also disclosed is a method of treating or inhibit islet immunity and/or T1D in a subject that involves administering to the subject a composition having a therapeutically effective amount of a myeloperoxidase (MPO) agonist. In some embodiments, the method involves measuring the levels of MPO in a sample from the subject. In these embodiments, the MPO agonist can be administered to subjects if the sample from the subject has MPO levels lower than control levels.

Also disclosed is a method of treating or inhibit islet immunity and/or T1D in a subject that involves administering to the subject a composition having a therapeutically effective amount of a MCP agonist. In some embodiments, the method involves measuring the levels of MCP in a sample from the subject. In these embodiments, the MCP agonist can be administered to subjects if the sample from the subject has MCP levels lower than control levels.

Also disclosed is a method of treating or inhibit islet immunity and/or T1D in a subject that involves administering to the subject a composition having a therapeutically effective amount of a IL-8 agonist. In some embodiments, the method involves measuring the levels of IL-8 in a sample from the subject. In these embodiments, the IL-8 agonist can be administered to subjects if the sample from the subject has IL-8 levels lower than control levels.

In preferred embodiments of these methods, the sample is blood, plasma, or serum isolated from the subject.

1. Recombinant Protein

In some embodiments, the IL-1ra, MIP-1β, IL-8, MCP-1, or MPO agonist is a recombinant protein. A recombinant protein is a protein that produced from recombinant DNA. Recombinant DNA is created by combining two or more sequences that would not normally occur together. For example, a recombinant protein is often produced by constructing an expression vector containing a nucleic acid sequence encoding the protein operably linked to an expression control sequence, such as a heterologous promoter. The expression vector generally contains heterologous sequences that promote replication of the vector within a cell system, such as bacteria. The coding region is often acquired by reverse transcribing mRNA for the protein. Therefore, the coding region in the recombinant DNA usually does not contain introns.

Thus, in some embodiments, IL-1ra agonist is recombinant IL-1ra. For example, the IL-1ra agonist can be recombinant human IL-1ra. Therefore, in certain embodiments, the IL-1ra agonist is anakinra (Kineret®), which is a recombinant, non-glycosylated version of human IL-1ra prepared from cultures of genetically modified Escherichia coli using recombinant DNA technology. It is made up of 153 amino acids and has a molecular weight of 17,257.6 g/mol (approx. 17.3 kilodaltons). Anakinra differs from native human IL-1ra in that it has the addition of a single methionine residue on its amino terminus. This drug is sold under the tradename Kineret® (Amgen) on the indication adult rheumatoid arthritis. For this indication, it is generally delivered as injection concentrate containing 100 mg each single dose.

In some embodiments, MIP-1β agonist is recombinant MIP-1β. For example, the MIP-1β agonist can be recombinant human MIP-1β. In some embodiments, MPO agonist is recombinant MPO. For example, the MPO agonist can be recombinant human MPO. In some embodiments, MCP agonist is recombinant MCP. For example, the MCP agonist can be recombinant human MCP. In some embodiments, IL-8 agonist is recombinant IL-8. For example, the IL-8 agonist can be recombinant human IL-8.

2. Nucleic Acids

In some embodiments, the IL-1ra, MIP-1β, IL-8, MCP-1, or MPO agonist is a nucleic acid encoding recombinant IL-1ra, MIP-1β, IL-8, MCP-1, or MPO. There are a number of compositions and methods which can be used to deliver nucleic acids to cells, either in vitro or in vivo. These methods and compositions can largely be broken down into two classes: viral based delivery systems and non-viral based delivery systems. For example, the nucleic acids can be delivered through a number of direct delivery systems such as, electroporation, lipofection, calcium phosphate precipitation, plasmids, viral vectors, viral nucleic acids, phage nucleic acids, phages, cosmids, or via transfer of genetic material in cells or carriers such as cationic liposomes.

In some embodiments, the IL-1ra, MIP-1β, IL-8, MCP-1, or MPO agonist is a transfer vector containing nucleic acid encoding recombinant IL-1ra, MIP-1β, IL-8, MCP-1, or MPO. Transfer vectors can be any nucleotide construction used to deliver genes into cells (e.g., a plasmid), or as part of a general strategy to deliver genes, e.g., as part of recombinant retrovirus or adenovirus. For example, the transfer vector can be a plasmid or viral vectors that transport the disclosed nucleic acids into the cell without degradation and include a promoter yielding expression of the gene in the cells into which it is delivered.

Viral vectors include, for example, Adenovirus, Adeno-associated virus, Herpes virus, Vaccinia virus, Polio virus, AIDS virus, neuronal trophic virus, Sindbis and other RNA viruses, including these viruses with the HIV backbone. Also preferred are any viral families which share the properties of these viruses which make them suitable for use as vectors. Retroviruses include Murine Maloney Leukemia virus, MMLV, and retroviruses that express the desirable properties of MMLV as a vector. Retroviral vectors are able to carry a larger genetic payload, i.e., a transgene or marker gene, than other viral vectors, and for this reason are a commonly used vector. Adenovirus vectors are relatively stable and easy to work with, have high titers, and can be delivered in aerosol formulation, and can transfect non-dividing cells. Pox viral vectors are large and have several sites for inserting genes, they are thermostable and can be stored at room temperature.

For example, the IL-1ra agonist can be a transfer vector containing a gene encoding recombinant IL-1ra operably linked to an expression control sequence. For example, the gene encoding recombinant IL-1ra can contain the sequence set forth in Accession No. NG_{—}021240. The MIP-1β agonist can be a transfer vector containing a gene encoding recombinant MIP-1β operably linked to an expression control sequence. For example, the gene encoding recombinant MIP-1β can contain the sequence set forth in Accession No. NM_{—}002984. The IL-8 agonist can be a transfer vector containing a gene encoding recombinant IL-8 operably linked to an expression control sequence. For example, the gene encoding recombinant IL-8 can contain the sequence set forth in Accession No. NM_{—}000584. The MCP-1 agonist can be a transfer vector containing a gene encoding recombinant MCP-1 operably linked to an expression control sequence. For example, the gene encoding recombinant MCP-1 can contain the sequence set forth in Accession No. NG_{—}012123. The MPO agonist can be a transfer vector containing a gene encoding recombinant MPO operably linked to an expression control sequence. For example, the gene encoding recombinant MPO can contain the sequence set forth in Accession No. NM_{—}000250.

The expression control sequence can contain a constitutive promoter, a tissue specific promoter, an inducible promoter, or a combination thereof. The gene encoding recombinant IL-1ra can be operably linked to other nucleic acid sequences.

For example, the expression control sequence can encode a fusion protein. A recombinant fusion protein is a protein created through genetic engineering of a fusion gene. This typically involves removing the stop codon from a cDNA sequence coding for the first protein, then appending the cDNA sequence of the second protein in frame through ligation or overlap extension PCR. That DNA sequence will then be expressed by a cell as a single protein.

The protein can be engineered to include the full sequence of both original proteins, or only a portion of either.

If the two entities are proteins, often linker (or “spacer”) peptides are also added which make it more likely that the proteins fold independently and behave as expected. Especially in the case where the linkers enable protein purification, linkers in protein or peptide fusions are sometimes engineered with cleavage sites for proteases or chemical agents which enable the liberation of the two separate proteins.

Alternatively, internal ribosome entry sites (IRES) elements can be used to create multigene, or polycistronic, messages. IRES elements are able to bypass the ribosome scanning model of 5′ methylated Cap dependent translation and begin translation at internal sites. Multiple genes can be efficiently expressed using a single promoter/enhancer to transcribe a single message.

3. Expression-Inducing Agents

In some embodiments, the IL-1ra, MIP-1β, IL-8, MCP-1, or MPO agonist is a molecule or other agent that induces endogenous expression of IL-1ra, MIP-1β, IL-8, MCP-1, or MPO.

For example, interferon-β has been shown to induce IL-1ra expression in human monocytes. Leptin can similarly induce the expression and secretion of IL-1ra in monocytic cells. TGF-β1 induces IL-1ra expression in vascular smooth muscle cells. Other proteins or synthetic agents that can promote IL-1ra expression or activity are known or can be similarly identified by one of skill in the art. Similarly, interferon-α treatment has been shown to increase at least MIP-1β and MCP-1 levels in patients with chronic hepatitis C virus (HCV) infection.

4. Protein Mimics

In some embodiments, the IL-1ra, MIP-1β, IL-8, MCP-1, or MPO agonist is a modified peptide or peptidomirnetic that mimics IL-1ra, MIP-1β, IL-8, MCP-1, or MPO. Amino acid analogs, peptide analogs, and peptidomimetics often have enhanced or desirable properties, such as, more economical production, greater chemical stability, enhanced pharmacological properties (half-life, absorption, potency, efficacy, etc.), reduced antigenicity, and others. Peptidomimetics are molecules that mimic peptide structure and have general features analogous to their parent polypeptides, such as the ability to bind the target substrate.

For example, there are numerous D amino acids or amino acids which have a different functional substituent than natural amino acids. The opposite stereo isomers of naturally occurring peptides are disclosed, as well as the stereo isomers of peptide analogs. These amino acids can readily be incorporated into polypeptide chains by charging tRNA molecules with the amino acid of choice and engineering genetic constructs that utilize, for example, amber codons, to insert the analog amino acid into a peptide chain in a site specific way. D-amino acids can be used to generate more stable peptides, because D amino acids are not recognized by peptidases and such. Systematic substitution of one or more amino acids of a consensus sequence with a D-amino acid of the same type (e.g., D-lysine in place of L-lysine) can be used to generate more stable peptides. Cysteine residues can be used to cyclize or attach two or more peptides together. This can be beneficial to constrain peptides into particular conformations.

Molecules can also be produced that resemble peptides, but which are not connected via a natural peptide linkage. For example, linkages for amino acids or amino acid analogs can include CH₂NH—, —CH₂S—, —CH₂—CH₂—, —CH═CH— (cis and trans), —COCH₂—, —CH(OH)CH₂—, and —CHH₂SO—. It is understood that peptide analogs can have more than one atom between the bond atoms, such as b-alanine, g-aminobutyric acid, and the like.

In some embodiments, the IL-1ra, MIP-1β, IL-8, MCP-1, or MPO agonist is an anti-idiotypic antibody. For example, the IL-1ra agonist can be an anti-idiotypic antibody that specifically binds one or more idiotopes within the paratope of an anti-IL-1ra antibody. The MIP-1β agonist can be an anti-idiotypic antibody that specifically binds one or more idiotopes within the paratope of an anti-MIP-1β antibody. The IL-8 agonist can be an anti-idiotypic antibody that specifically binds one or more idiotopes within the paratope of an anti-IL-8 antibody. The MCP-1 agonist can be an anti-idiotypic antibody that specifically binds one or more idiotopes within the paratope of an anti-MCP-1 antibody. The MPO agonist can be an anti-idiotypic antibody that specifically binds one or more idiotopes within the paratope of an anti-MPO antibody.

A “paratope” is the part of an antibody that specifically interacts with the epitope of the antigen. An “idiotope” is an antigenic determinant (epitope) within the variable region of the immunoglobulin product of a clone. Thus, idiotopes are epitopes to which an anti-idiotype antibody binds. The paratope of the anti-idiotypic antibody contains a mirror image of the original antibody's paratope, which is a mirror image of the antigen's epitope. Consequently, the anti-idiotypic antibody contains a three-dimensional binding site that mimics the structure of the original antigen.

F. Using Antagonists

A method of treating or inhibit islet immunity and/or T1D in a subject is disclosed that involves administering to the subject a composition having a therapeutically effective amount of a serum amyloid (SAA) antagonist. In some embodiments, the method involves measuring the levels of SAA in a sample from the subject. In these embodiments, the SAA antagonist can be administered to subjects if the sample from the subject has elevated SAA levels compared to a control.

Also disclosed is a method of treating or inhibit islet immunity and/or T1D in a subject that involves administering to the subject a composition having a therapeutically effective amount of an IGFBP2 antagonist. In some embodiments, the method involves measuring the levels of IGFBP2 in a sample from the subject. In these embodiments, the IGFBP2 antagonist can be administered to subjects if the sample from the subject has elevated IGFBP2 levels compared to a control.

Also disclosed is a method of treating or inhibit islet immunity and/or T1D in a subject that involves administering to the subject a composition having a therapeutically effective amount of an Adiponectin antagonist. In some embodiments, the method involves measuring the levels of Adiponectin in a sample from the subject. In these embodiments, the Adiponectin antagonist can be administered to subjects if the sample from the subject has elevated Adiponectin levels compared to a control.

In preferred embodiments of these methods, the sample is blood, plasma, or serum isolated from the subject.

1. Therapeutic Antibody

In some embodiments, SAA antagonist is a therapeutic antibody that selectively targets SAA. For example, the therapeutic SAA antibody can be a single-chain antibody, antibody fragment, or other antibody variant capable of binding to SAA and preventing its activity within a cell.

In some embodiments, IGFBP2 antagonist is a therapeutic antibody that selectively binds IGFBP2. For example, the therapeutic IGFBP2 antibody can be a single-chain antibody, antibody fragment, or other antibody variant capable of binding to IGFBP2 and preventing its activity within a cell.

In some embodiments, adiponectin antagonist is a therapeutic antibody that selectively binds I adiponectin. For example, the therapeutic adiponectin antibody can be a single-chain antibody, antibody fragment, or other antibody variant capable of binding to adiponectin and preventing its activity within a cell.

The term “antibodies” is used herein in a broad sense and includes both polyclonal and monoclonal antibodies. In addition to intact immunoglobulin molecules, also included in the term “antibodies” are fragments or polymers of those immunoglobulin molecules, and human or humanized versions of immunoglobulin molecules or fragments thereof, as long as they are chosen for their ability to interact with SAA, IGFBP2, or adiponectin such that SAA, IGFBP2, or adiponectin activity is inhibited.

By “selectively binds” is meant that an antibody recognizes and physically interacts with its cognate antigen (e.g., a SAA, IGFBP2, or Adiponectin polypeptide) and does not significantly recognize and interact with other antigens; such an antibody may be a polyclonal antibody or a monoclonal antibody, which are generated by techniques that are well known in the art.

2. Chemical Agents

In some embodiments, the therapeutic agent can be a small chemical compound that can inhibit the expression of SAA.

In some embodiments, the therapeutic agent can be a small chemical compound that can inhibit the expression of IGFBP2.

In some embodiments, the therapeutic agent can be a small chemical compound that can inhibit the expression of Adiponectin.

3. Functional Nucleic Acids

The SAA, IGFBP2, or adiponectin antagonist of the provided method can be a functional nucleic acid, such as an antisense molecule, triplex forming molecule, RNAi, or external guide sequence.

Antisense molecules are designed to interact with a target nucleic acid molecule through either canonical or non-canonical base pairing. The interaction of the antisense molecule and the target molecule is designed to promote the destruction of the target molecule through, for example, RNAseH mediated RNA-DNA hybrid degradation. Alternatively the antisense molecule is designed to interrupt a processing function that normally would take place on the target molecule, such as transcription or replication. Antisense molecules can be designed based on the sequence of the target molecule. Numerous methods for optimization of antisense efficiency by finding the most accessible regions of the target molecule exist. It is preferred that antisense molecules bind the target molecule with a dissociation constant (K_{d}) less than or equal to 10⁻⁶, 10⁻⁸, 10⁻¹⁰, or 10⁻¹².

Triplex forming functional nucleic acid molecules are molecules that can interact with either double-stranded or single-stranded nucleic acid. When triplex molecules interact with a target region, a structure called a triplex is formed, in which there are three strands of DNA forming a complex dependant on both Watson-Crick and Hoogsteen base-pairing. Triplex molecules are preferred because they can bind target regions with high affinity and specificity. It is preferred that the triplex forming molecules bind the target molecule with a K_{d} less than 10⁻⁶, 10⁻⁸, 10⁻¹⁰, or 10⁻¹².

External guide sequences (EGSs) are molecules that bind a target nucleic acid molecule forming a complex, and this complex is recognized by RNase P, which cleaves the target molecule. EGSs can be designed to specifically target a RNA molecule of choice. RNAse P aids in processing transfer RNA (tRNA) within a cell. Bacterial RNAse P can be recruited to cleave virtually any RNA sequence by using an EGS that causes the target RNA:EGS complex to mimic the natural tRNA substrate. Similarly, eukaryotic EGS/RNAse P-directed cleavage of RNA can be utilized to cleave desired targets within eukarotic cells.

Gene expression can also be effectively silenced in a highly specific manner through RNA interference (RNAi). This silencing was originally observed with the addition of double stranded RNA (dsRNA). Once dsRNA enters a cell, it is cleaved by an RNase III-like enzyme, Dicer, into double stranded small interfering RNAs (siRNA) 21-23 nucleotides in length that contains 2 nucleotide overhangs on the 3′ ends. In an ATP dependent step, the siRNAs become integrated into a multi-subunit protein complex, commonly known as the RNAi induced silencing complex (RISC), which guides the siRNAs to the target RNA sequence. At some point the siRNA duplex unwinds, and it appears that the antisense strand remains bound to RISC and directs degradation of the complementary mRNA sequence by a combination of endo and exonucleases. However, the effect of iRNA or siRNA or their use is not limited to any type of mechanism.

Short Interfering RNA (siRNA) is a double-stranded RNA that can induce sequence-specific post-transcriptional gene silencing, thereby decreasing or even inhibiting gene expression. In one example, an siRNA triggers the specific degradation of homologous RNA molecules, such as mRNAs, within the region of sequence identity between both the siRNA and the target RNA. Sequence specific gene silencing can be achieved in mammalian cells using synthetic, short double-stranded RNAs that mimic the siRNAs produced by the enzyme. siRNA can be chemically or in vitro-synthesized or can be the result of short double-stranded hairpin-like RNAs (shRNAs) that are processed into siRNAs inside the cell. Synthetic siRNAs are generally designed using algorithms and a conventional DNA/RNA synthesizer. Suppliers include Ambion (Austin, Tex.), ChemGenes (Ashland, Mass.), Dharmacon (Lafayette, Colo.), Glen Research (Sterling, Va.), MWB Biotech (Esbersberg, Germany), Proligo (Boulder, Colo.), and Qiagen (Vento, The Netherlands). siRNA can also be synthesized in vitro using kits such as Ambion's SILENCER® siRNA Construction Kit. Disclosed herein are any siRNA designed as described above based on the sequences for SAA, IGFBP2, or Adiponectin.

The production of siRNA from a vector is more commonly done through the transcription of a short hairpin RNAs (shRNAs). Kits for the production of vectors comprising shRNA are available, such as, for example, Imgenex's GENESUPPRESSOR™ Construction Kits and Invitrogen's BLOCK-IT™ inducible RNAi plasmid and lentivirus vectors.

4. Dominant Negative and Competitive Inhibitors

The SAA, IGFBP2, or Adiponectin antagonist of the provided method can be a competitive inhibitor that binds SAA, IGFBP2, or Adiponectin and prevents the protein from binding its target substrate. Competitive inhibition is a form of enzyme inhibition where binding of the inhibitor to the active site on the enzyme prevents binding of the enzyme to its substrate. In some embodiments, the competitive inhibitors bind in the same binding site as the target substrate. In other embodiments, the competitive inhibitor binds to an allosteric site of the enzyme and prevents target substrate binding. Therefore, competitive inhibitor inhibitors of SAA, IGFBP2, and Adiponectin can be designed using routine skill based on knowledge of the target substrates for SAA, IGFBP2, or Adiponectin. For example, truncated proteins or peptide fragments can be produced containing the binding region of SAA, IGFBP2, or Adiponectin substrate, which would compete for the binding site on SAA, IGFBP2, or Adiponectin, respectively.

The SAA, IGFBP2, or Adiponectin antagonist of the provided method can be a dominant negative protein that competes with SAA, IGFBP2, or Adiponectin for binding to their targets. Dominant negative proteins contain the binding domain of the native protein that allows it to bind the target substrate but lacks one more functional domains (or contain a mutation inactivating a functional domain). These dominant negative proteins compete with wild type protein for binding to the target substrate and thereby reduce their activity. Therefore, dominant negative inhibitors of SAA, IGFBP2, and Adiponectin can be designed based on knowledge available in the art pertaining to protein structure, specifically knowledge relating to the regions involved in binding their target substrates and regions necessary for subsequent activity.

### VI. Pharmaceutical Composition

The compositions disclosed can be used therapeutically in combination with a pharmaceutically acceptable carrier. The carrier would naturally be selected to minimize any degradation of the active ingredient and to minimize any adverse side effects in the subject, as would be well known to one of skill in the art.

The materials may be in solution, suspension (for example, incorporated into microparticles, liposomes, or cells). These may be targeted to a particular cell type via antibodies, receptors, or receptor ligands. Vehicles such as “stealth” and other antibody conjugated liposomes, receptor mediated targeting of DNA through cell specific ligands, lymphocyte directed tumor targeting, and highly specific therapeutic retroviral targeting of murine glioma cells in vivo. In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration.

Pharmaceutical carriers are known to those skilled in the art. These most typically would be standard carriers for administration of drugs to humans, including solutions such as sterile water, saline, and buffered solutions at physiological pH. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Peptides may be chemically modified so that oral delivery of the derivative is efficacious. Generally, the chemical modification contemplated is the attachment of at least one moiety to the component molecule itself, where said moiety permits (a) inhibition of proteolysis; and (b) uptake into the blood stream from the stomach or intestine. Also desired is the increase in overall stability of the component or components and increase in circulation time in the body. For example, PEGylation is a preferred chemical modification for pharmaceutical usage. Other moieties that may be used include: propylene glycol, copolymers of ethylene glycol and propylene glycol, carboxymethyl cellulose, dextran, polyvinyl alcohol, polyvinyl pyrrolidone, polyproline, poly-1,3-dioxolane and poly-1,3,6-tioxocane.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amities and substituted ethanolamines.

A. Therapeutic Administration

The disclosed compositions, including pharmaceutical composition, may be administered in a number of ways depending on whether local or systemic treatment is desired, and on the area to be treated. For example, the disclosed compositions can be administered intravenously, intraperitoneally, intramuscularly, subcutaneously, intracavity, or transdermally. The compositions may be administered orally, parenterally (e.g., intravenously), by intramuscular injection, by intraperitoneal injection, transdermally, extracorporeally, ophthalmically, vaginally, rectally, intranasally, topically or the like, including topical intranasal administration or administration by inhalant.

Parenteral administration of the composition, if used, is generally characterized by injection. Injectables can be prepared in conventional forms, either as liquid solutions or suspensions, solid forms suitable for solution of suspension in liquid prior to injection, or as emulsions. A revised approach for parenteral administration involves use of a slow release or sustained release system such that a constant dosage is maintained. See, e.g., U.S. Pat. No. 3,610,795, which is incorporated by reference herein.

The disclosed compositions may be administered prophylactically to T1D patients or subjects who are at risk for T1D. Thus, the method can further involve identifying a subject at risk for T1D prior to administration of the disclosed compositions.

The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the disclosed teachings. For example, effective dosages and schedules for administering the compositions may be determined empirically, and making such determinations is within the skill in the art. The dosage ranges for the administration of the compositions are those large enough to produce the desired effect in which the symptoms disorder are effected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389. A typical daily dosage of the antibody used alone might range from about 1 μg/kg to up to 100 mg/kg of body weight or more per day, depending on the factors mentioned above.

### EXAMPLES

### Example 1

### Reduced IL-1Ra in T1D Patients

Methods

Patients and Samples

The first dataset (DISCOVER) consisted of 682 autoantibody-negative (AbN) controls and 697 T1D patients, while the second dataset (CONFIRM) included 1696 AbN controls and 1586 T1D patients. The subjects in these two datasets were participants in the prospective assessment in newborns of diabetes autoimmunity (PANDA) study. All subjects were Caucasians living in the State of Georgia and randomly selected from the PANDA cohort. The characteristics for the subjects are summarized in Table 1.

TABLE 1
Demographic and laboratory information on the cross-sectional
datasets.

| | | |
|---|---|---|
| AbN | T1D | P-value |

DISCOVER dataset

| | | | | |
|---|---|---|---|---|
| Sex | Female | 363 | 370 | 0.99 |
| | Male | 319 | 327 | |
| HLA Genotype | 0201/0201 | 55 | 60 | 5.5 × 10⁻²² |
| | 0302/0302 | 40 | 52 | |
| | 0201/0302 | 92 | 213 | |
| | 0201/x | 137 | 79 | |
| | 0302/x | 153 | 149 | |
| | x/x | 152 | 54 | |
| Age (years) | | 22.9 ± 18.3 | 29.1 ± 18.9 | 8.1 × 10⁻¹⁰ |
| Duration of T1D | | | 13.0 ± 14.1 | |
| (yrs) | | | | |

CONFIRM dataset

| | | | | |
|---|---|---|---|---|
| Sex | Female | 932 | 800 | 0.01 |
| | Male | 764 | 786 | |
| HLA Genotype | 0201/0201 | 126 | 140 | 1.1 × 10⁻⁴⁷ |
| | 0302/0302 | 98 | 114 | |
| | 0201/0302 | 213 | 443 | |
| | 0201/x | 370 | 323 | |
| | 0302/x | 379 | 233 | |
| | x/x | 401 | 141 | |
| Age (years) | | 22.7 ± 18.5 | 31.5 ± 19.0 | <10⁻¹⁶ |
| Duration of T1D | | | 14.9 ± 13.9 | |
| (yrs) | | | | |

Written informed consent was obtained from all subjects or their parents/guardians. Biological samples (DNA and serum) along with demographic and clinical data were collected from each study subject. Serum samples were collected, processed and stored within less than 2 hours. Repeated freeze/thaw cycles were avoided for all samples.

Serum Protein Assays

IL-1ra in serum was measured using a Luminex® kit (Millipore, Billerica, Mass., USA) according to manufacturer's protocol. Briefly, the kit is based on sandwich immunoassay, which consists of dyed microspheres conjugated with a monoclonal antibody specific for S0041A as a capture antibody. Serum samples were incubated with the antibody-coupled microspheres after they were incubated with biotinylated detection antibody before the addition of streptavidin-phycoerythrin. The captured bead-complexes were then read by a FlexMap-3D system (Millipore, Billerica, Mass., USA) with the following instrument settings: events/bead: 50, minimum events: 0, Flow rate: 60 μl/min, Sample size: 50 μl, discriminator gate: 12800. For all the assays median fluorescence intensity (MFI) were collected.

IL-1ra level (concentration) for each subject was estimated using a linear regression fit to the standard curve of recombinant IL-1ra protein standards included on each plate using a 3-fold serial dilution series. The log of the observed median fluorescence intensity (MFI) for the dilution series samples were regressed on the log of the known concentration for these samples. The concentration of subject samples with an MFI that was either above the largest MFI or below the smallest MFI of the known standards were set to the estimated concentration for the appropriate extreme known standards. Samples with a CV among microspheres within the well that was greater than 100 were removed since the MFI for such wells are unreliable. The estimated IL-1ra concentrations were on the log scale, and as such, all analyses were conducted using ln(IL-1ra concentration). Estimation of IL-1ra concentration using standard curves were conducted using R.

Data Analysis

The potential difference between T1D patients, AbN controls from the general population (GP), first degree relatives (FDR) controls in the DISCOVER dataset was initially examined using a t-test. Because there was no significant difference between the two control subsets, all AbN samples were combined for subsequent analysis. To rule out the effects of age, sex and HLA genotype, the samples were matched for these three variable to create a paired dataset. Conditional logistic regression was then performed with single protein or a combination of proteins to detect the odd or having T1D. serum levels of proteins were treated as linear variable or divided into quartiles to be used as a categorical vector. Effect of age on serum levels of proteins for various risk groups based on HLA genotype was tested using linear regression.

As with the DISCOVER dataset, the difference between AbN control groups and T1D patients in the CONFIRM dataset was initially examined using a t-test. A regression model was next used to examine the odds of having T1D in a paired dataset, matched for age, sex and HLA genotypes.

Results

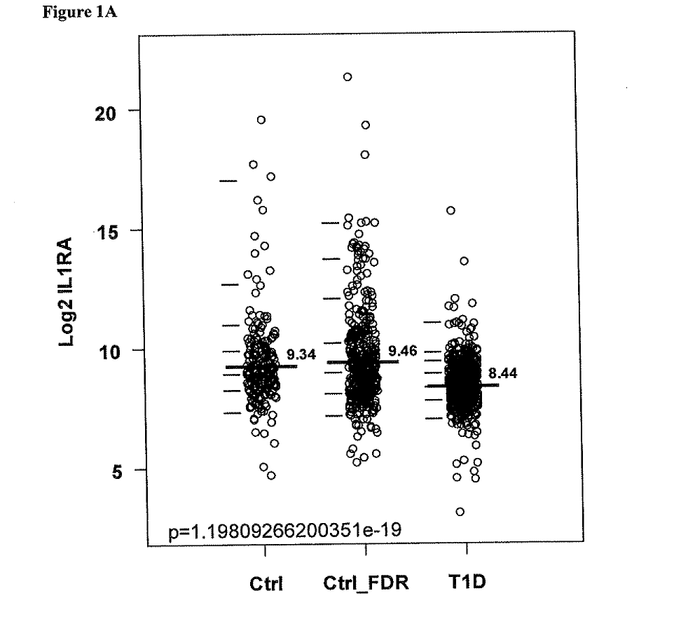

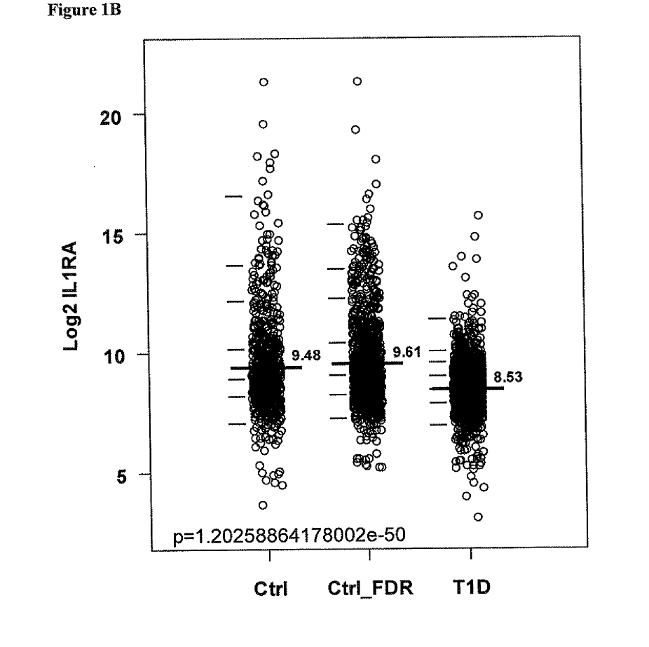

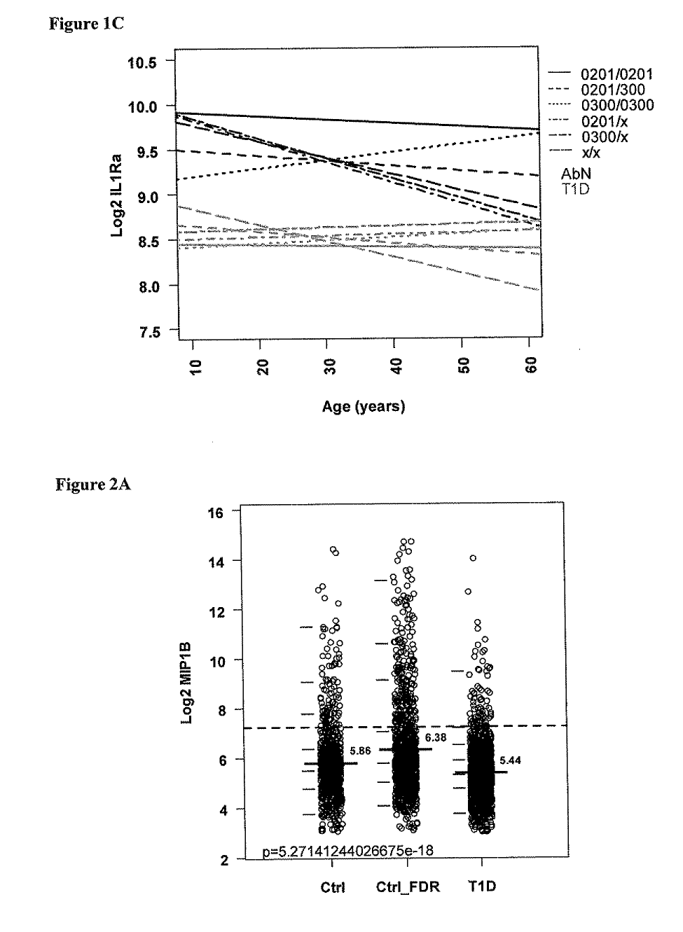

In an attempt to identify T1D biomarkers, 61 serum proteins including various cytokines, chemokines and soluble receptors were measured using multiplex Luminex® assays in the sera of 697 T1D and 682 AbN subjects in the DISCOVER sample set. Paired t-test revealed a 2.22-fold reduction of serum IL-1ra in T1D patients compared to controls (p=2.1×10⁻¹⁵). Serum IL-1ra level for each patient and control is presented in FIG. 1A. To confirm the results, serum IL-1ra was measured for 1696 controls and 1586 patients in the CONFIRM sample set (FIG. 1B). A similar 2.0-fold reduction in serum IL-1ra was found in patients compared to controls (p<10⁻¹⁶) with the difference again remaining highly significant after matching for sex, age, and HLA (p=5.3×10⁻¹⁰). However, the difference between controls and patients depended on age and HLA (p=8.8×10⁻⁹ for age*subject group interaction, p=2.1×10⁻⁵ for HLA*subject group interaction). Controls exhibited decreasing IL1-ra levels with increasing age, while patients showed no changes in relationship (FIG. 1C). The high-risk HLA genotypes had the highest IL1-ra levels in controls, while the high-risk genotypes had the lowest IL-1ra levels in patients (FIG. 1C).

### Example 2

### Serum Protein Changes in T1D Patients are Associated with Increased Risk of T1D

Results

To evaluate the risk of T1D logistic regression was used to analyze the serum concentration of proteins as a linear variable. The analysis suggested that lower serum levels of IL-1Ra, MIP1b, IL8, MCP-1 and MPO has an very high odds of having T1D (Table 2). similarly increase serum levels of SAA, IGFBP2 and ADIPOQ has an increased risk of having T1D (Table 2),

TABLE 2
Odds ratio of having T1D, using serum proteins as linear variable

| Protein | OR | p-val |
|---|---|---|
| IL1Ra | 0.58 (0.5-0.68) | 1.00E−12 |
| MIP1B | 0.74 (0.66-0.83) | 4.70E−07 |
| IL8 | 0.65 (0.58-0.73) | 8.30E−13 |
| MCP1 | 0.63 (0.54-0.72) | 5.60E−11 |
| SAA | 1.15 (1.02-1.29) | 0.018 |
| IGFBP2 | 2.1 (1.82-2.42) | 8.20E−25 |
| ADIPOQ | 1.95 (1.73-2.19) | 1.00E−27 |
| MPO | 0.51 (0.46-0.56) | 4.00E−42 |

Next the serum samples were analyzed by converting the serum concentration of IL-1ra, MIP-1b, IL8, MCP-1, SAA, IGFBP2, ADIPOQ and MPO into 4 categories based on quartiles. Logistic regression was then performed by comparing the 1^{st} quartile with the other three quartiles. The analysis suggests that subjects in 4^{th} quartile has very high odds of having T1D (Table 3).

TABLE 3
Odds ratio of having T1D, using protein concentrations as categorical
variable.

| | OR | OR | OR | |
|---|---|---|---|---|
| | Second | Third | Fourth | |
| Protein | quartile | quartile | quartile | p-trend |
| IL1Ra | 0.84 | 0.45 | 0.08 | 3.48E−21 |
| | (0.63-1.13) | (0.32-0.63) | (0.05-0.14) | |
| MIP1B | 1.36 | 0.82 | 0.26 | 2.75E−08 |
| | (1.03-1.81) | (0.6-1.11) | (0.17-0.4) | |
| IL8 | 1.32 | 0.68 | 0.09 | 2.16E−15 |
| | (0.99-1.74) | (0.5-0.93) | (0.05-0.16) | |
| MCP1 | 1.13 | 0.68 | 0.4 | 1.90E−08 |
| | (0.85-1.5) | (0.5-0.92) | (0.28-0.56) | |
| SAA | 1.16 | 1.24 | 1.57 | 0.005130445 |
| | (0.85-1.57) | (0.9-1.71) | (1.14-2.15) | |
| IGFBP2 | 2.1 | 3.56 | 5.35 | 1.85E−23 |
| | (1.48-2.97) | (2.52-5.03) | (3.79-7.55) | |
| ADIPOQ | 1.53 | 1.62 | 6.29 | 6.00E−35 |
| | (1.22-1.92) | (1.27-2.07) | (4.75-8.33) | |
| MPO | 0.56 | 0.42 | 0.10 | 2.00E−46 |
| | (0.45-0.71) | (0.33-0.54) | (0.07-0.14) | |

### Example 3

### Serum Protein Changes in T1D Patients

Results

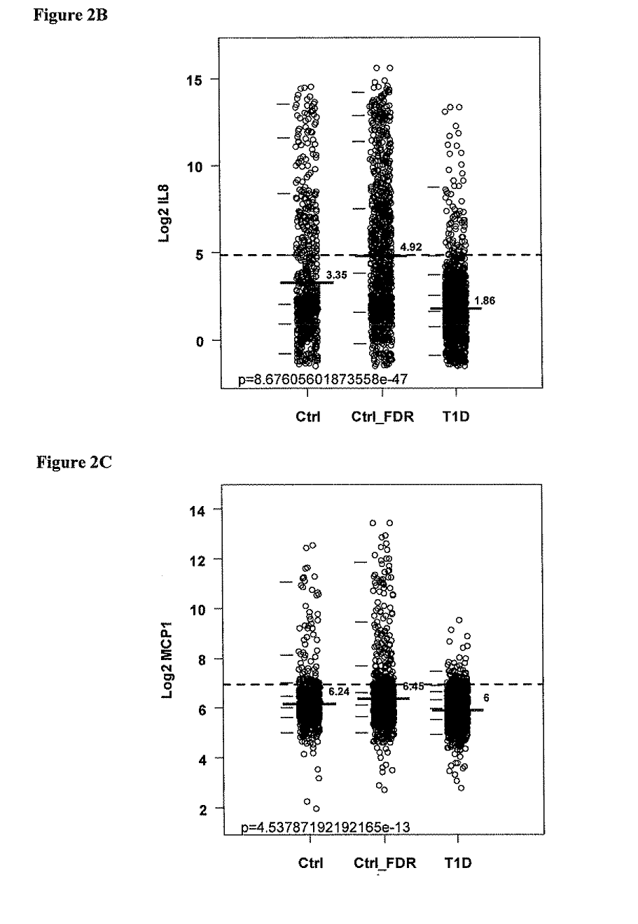

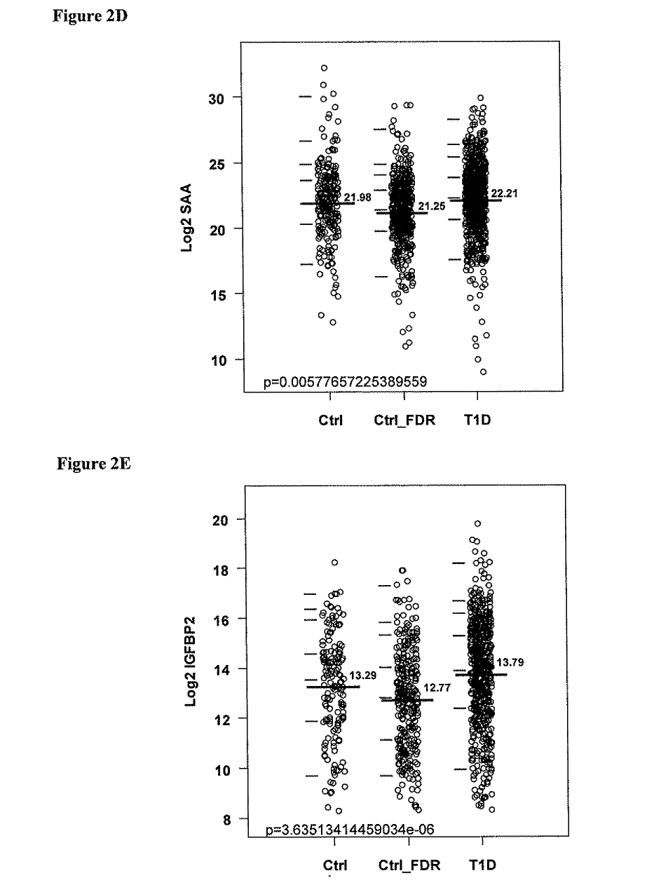

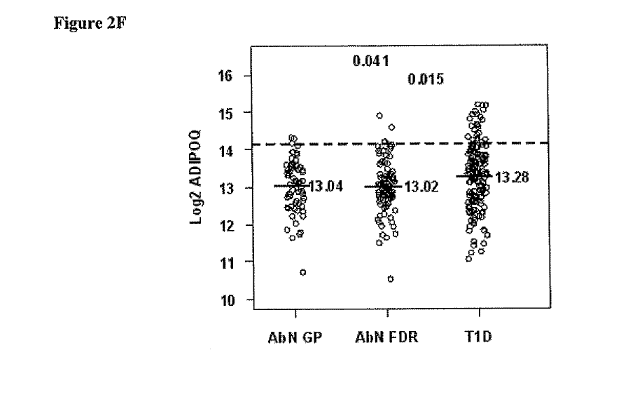

In an effort to identify serum biomarkers and elucidate the underlying disease mechanism for T1D, two different discovery platforms were used to identify serum protein changes in T1D patients. In the first platform, serum proteins were analyzed using 2D-HPLC and mass spectrometry technologies. In the second platform, multiplex Luminex® assays were used to analyze a large number of serum proteins, which might be implicated in the pathogenesis of various inflammatory diseases. These studies identified seven additional serum proteins (MIP-1b, IL8, MCP-1, MPO, SAA, IGFBP2, and ADIPOQ) that showed decreased or increased levels in T1D patients compared to healthy controls (FIG. 2). These results were based on a large cross sectional dataset with over 1500 patients and 1500 controls, thus providing evidence that these proteins may be used as biomarkers for T1D.

### Example 4

### Serum Myeloperoxidase (MPO) is Significantly Lower in Type 1 Diabetes Patients

Results

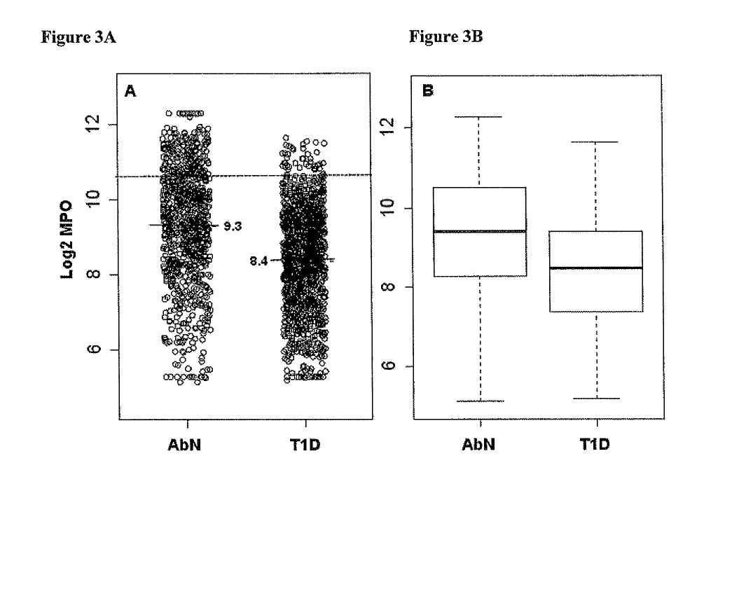

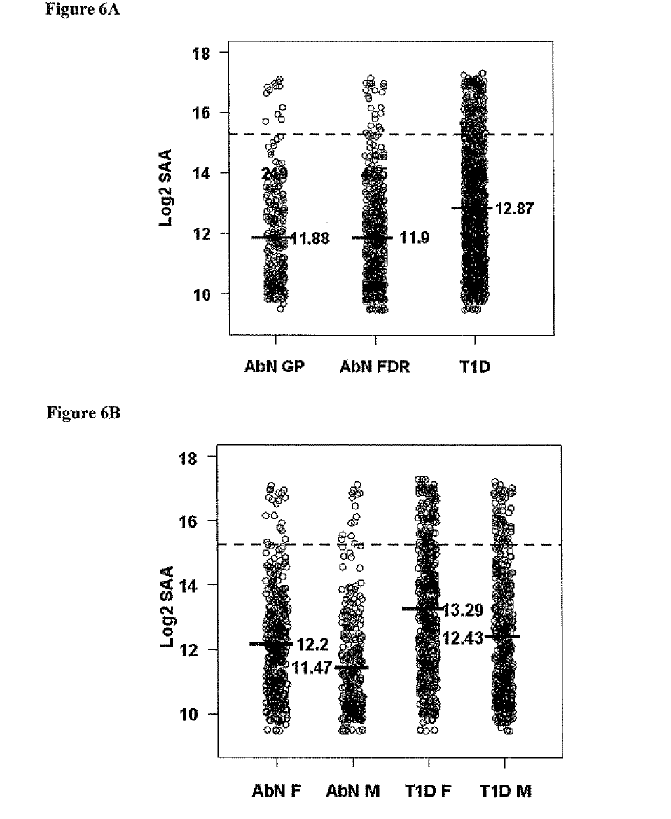

MPO was measured in 848 AbN controls and 1,139 T1D patients using Luminex® assays. T-tests were initially used to compare the differences in the mean levels of MPO between T1D and AbN groups. As illustrated in FIG. 6A, the mean MPO level was significantly lower in the T1D group (mean=333 ng/ml or log 2 mean=8.4) than the entire AbN control group (mean=626 ng/ml or log 2 mean=9.3) (FIG. 3), representing a mean reduction of approximately 2-fold in T1D patients (p<10⁻³⁶). The percentile distribution of MPO in T1D and AbN groups are presented using the box plot (FIG. 3B). The 95th percentile value of serum MPO in T1D subjects was also determined to be at 10.5 and was used as a cutoff to determine the distribution of subjects above or below this threshold. In the AbN control group, 24.7% of the subjects have MPO above the threshold compared to 5% of the T1D subjects, suggesting that higher MPO is associated with lower T1D risk (odds ratio=0.16, p=10⁻³⁶) (Table 4).

TABLE 4
Serum MPO in T1D and AbN controls.

| Group | Mean* | SD* | T1D/AbN* | p-value |
|---|---|---|---|---|
| T1D | 8.4 | 1.4 | | |
| AbN | 9.3 | 1.6 | 0.53 | 2 × 10⁻³⁷ |
| AbN FDR | 9.4 | 1.6 | 0.50 | 1 × 10⁻³³ |
| AbN GP | 9.1 | 1.7 | 0.60 | 2 × 10⁻¹² |
| T1D females | 8.4 | 1.4 | | |
| AbN females | 9.3 | 1.6 | 0.52 | 2 × 10⁻²² |
| T1D males | 8.4 | 1.4 | | |
| AbN males | 9.2 | 1.7 | 0.54 | 3 × 10⁻¹⁶ |

| Group | Above | Below | OR | LCL | UCL | p-value |
|---|---|---|---|---|---|---|
| T1D | 58 | 1081 | | | | |
| AbN | 209 | 639 | 0.16 | 0.15 | 0.17 | 1 × 10⁻³⁶ |
| AbN FDR | 142 | 387 | 0.15 | 0.13 | 0.17 | 4 × 10⁻³⁷ |
| AbN GP | 67 | 252 | 0.20 | 0.17 | 0.23 | 3 × 10⁻¹⁹ |
| T1D females | 25 | 569 | | | | |
| AbN females | 113 | 348 | 0.14 | 0.11 | 0.16 | 3 × 10⁻²² |
| T1D males | 33 | 512 | | | | |
| AbN males | 96 | 291 | 0.20 | 0.16 | 0.23 | 3 × 10⁻¹³ |

*Means and SDs were derived using log2 transformed MPO concentration. T1D/AbN ratio was obtained using 2(log2 mean of T1D − log 2 mean of AbN). LCL = lower confidence limit, UCL = upper confidence limit. Above and below indicates the number of subjects above or below the cutoff threshold 10.5 (the 95th percentile value of serum MPO in T1D subjects).

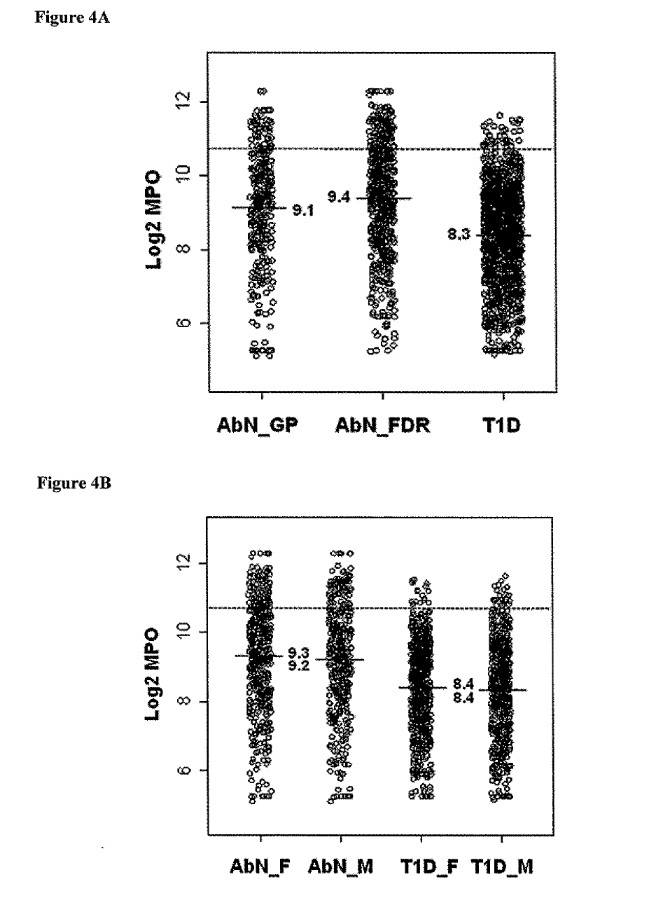

The means and distributions of serum MPO levels were very similar in both the FDR controls and GP controls (FIG. 4A). However, T1D subjects had lower serum MPO levels than both the FDR controls (mean=670 ng/ml or log 2 mean=9.4, p=1×10⁻³³) and the GP controls (mean=556 ng/ml or log 2 mean=9.1, p=2×10⁻¹²).

Since subject age, gender and duration of disease are potentially confounding variables that may be responsible for the observed differences between T1D and AbN subjects, the relationship of these variables and MPO level was examined. Age, gender and duration of diabetes were included in the regression analyses that included both T1D and AbN subjects. These analyses did not reveal any significant association between MPO and age, sex or duration of diabetes. The mean serum MPO levels were both significantly lower in female and male T1D groups than corresponding AbN groups (Table 4 and FIG. 4B). The mean serum MPO levels appeared to differ between T1D and AbN in a similar manner across all age groups.

### Example 5

### SAA is Increased in T1D Patients and Diabetic Complications

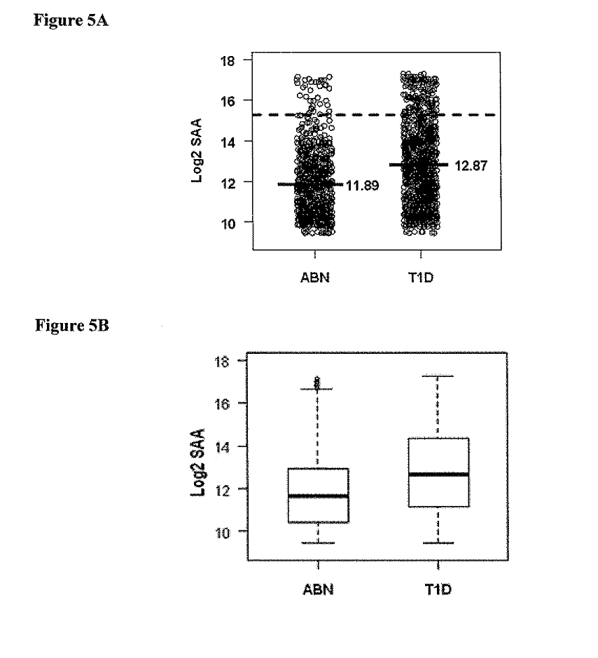

SAA was selected from the mass spectrometry-based discovery studies as a candidate for T1D biomarkers. The confirmation study included 995 T1D patients and 704 AbN controls with valid results. A simple student t-test suggested that the mean SAA level was significantly higher in the T1D group (mean=6,654 ng/ml or log 2 mean=12.7) than the AbN control group (mean=3,566 ng/ml or log 2 mean=11.8) (FIG. 5A, p<10⁻²⁵). The percentile distribution of SAA in T1D and AbN groups was presented using a box plot (FIG. 5B), which showed an upward shift of SAA levels in T1D subjects at the 25th, 50th and 75th percentile. The 95th percentile value of Log2 (SAA) in AbN subjects was determined to be 15.1 and 16% of the patients had SAA levels above this threshold compared to 5% of the AbN subjects (odds ratio=3.5, p=9×10⁻¹⁴) (Table 5), suggesting that there were more T1D subjects with very high SAA levels.

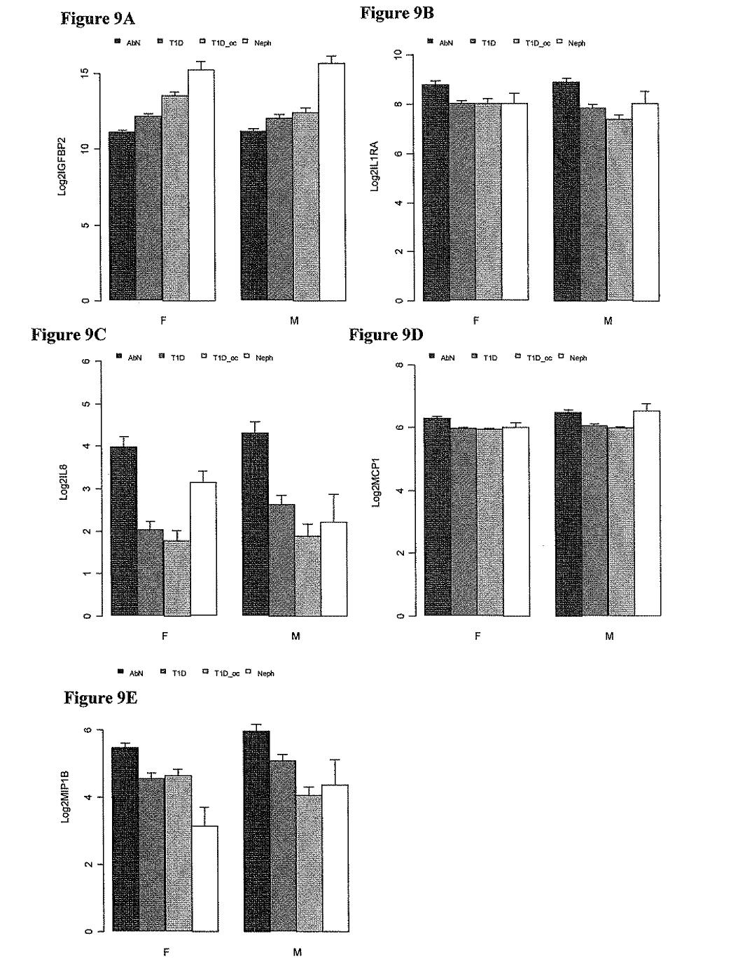

It was next examined whether SAA levels in FDR subjects differed from those in GP controls or T1D patients. As shown in FIG. 9A, FDR and GP controls had similar SAA levels, and T1D subjects had higher serum SAA levels than both the FDR controls (mean=3,557 ng/ml or log 2 mean=11.80, p=4×10⁻²⁰) and the GP controls (mean=3,500 ng/ml or log 2 mean=11.77, p=1×10⁻¹⁵) (Table 5).

TABLE 5
Serum SAA in T1D and AbN controls.

| Group | Above | Below | OR | LCL | UCL | p-value |
|---|---|---|---|---|---|---|
| T1D | 179 | 960 | | | | |
| AbN | 43 | 805 | 3.49 | 3.19 | 3.81 | 9 × 10⁻¹⁴ |
| AbN FDR | 29 | 500 | 3.21 | 2.77 | 3.74 | 4 × 10⁻⁹ |
| AbN GP | 14 | 305 | 4.06 | 3.49 | 4.72 | 1 × 10⁻⁷ |
| T1D females | 114 | 480 | | | | |
| AbN females | 28 | 433 | 3.67 | 3.07 | 4.39 | 6 × 10⁻¹⁰ |
| T1D males | 65 | 480 | | | | |
| AbN males | 15 | 372 | 3.36 | 2.81 | 4.01 | 2 × 10⁻⁵ |

| Group | Mean* | SE* | T1D/AbN* | p-value |
|---|---|---|---|---|
| T1D | 12.8 | 0.07 | | |
| AbN | 12.1 | 0.07 | 1.61 | 5 × 10⁻¹³ |
| AbN FDR | 12.1 | 0.09 | 1.61 | 2 × 10⁻¹⁰ |
| AbN GP | 12.1 | 0.12 | 1.61 | 4 × 10⁻⁷ |

*Means and SE were derived using log2 transformed SAA concentration. T1D/AbN ratio was obtained using 2^{(log2 mean of T1D - mean of AbN)}. LCL = lower confidence limit, UCL = upper confidence limit. Above and below indicates the number of subjects above or below the cutoff threshold 15.1 (the 95^{th} percentile value of serum SAA in AbN subjects).

Since subject age, gender and duration of disease are potentially confounding variables that could be responsible for the observed differences between T1D and AbN subjects, the relationship of these variables with SAA was examined. The distributions and mean SAA levels in male and female T1D and AbN subjects are shown in FIG. 6B. In both phenotypic groups, female subjects had significantly higher SAA levels than male subjects (overall sex effect in regression: p=1.1×10⁻¹⁶). Although females did have higher SAA levels, the difference between T1D patients and AbN controls did not differ between the sexes (sex and T1D phenotype interaction: p>0.05).

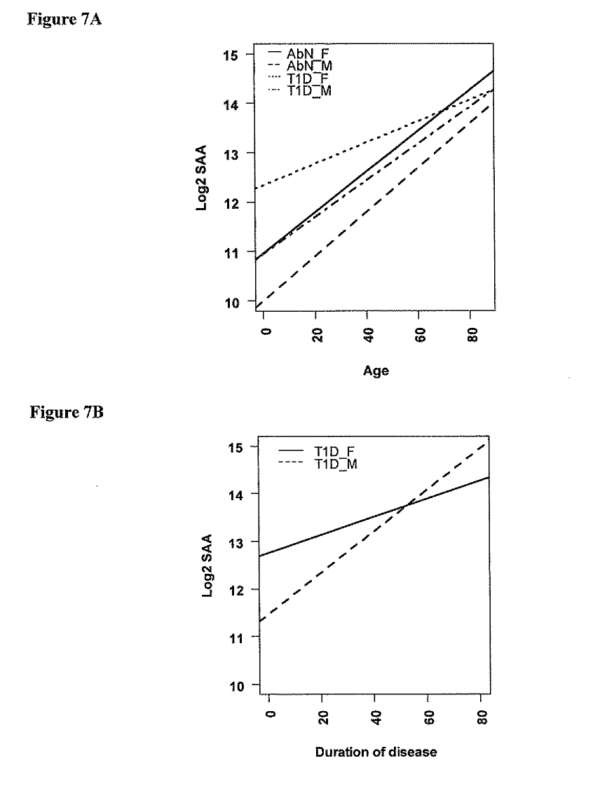

Mean SAA levels also increased with age in both the AbN control group (p=2.2×10⁻¹⁶) and T1D group (p=3.1×10⁻¹³, FIG. 7A). Furthermore, mean SAA levels increased significantly with duration of T1D, but the increase was much larger in male patients (p=6.1×10⁻⁷) than in female patients (p=0.027), with the difference being significant (p=0.0445; FIG. 7B).

Regression analyses including age and gender were therefore performed to assess the SAA difference between T1D and AbN control groups. T1D subjects had significantly higher SAA levels after adjusting for these covariates (p=5×10⁻¹³).

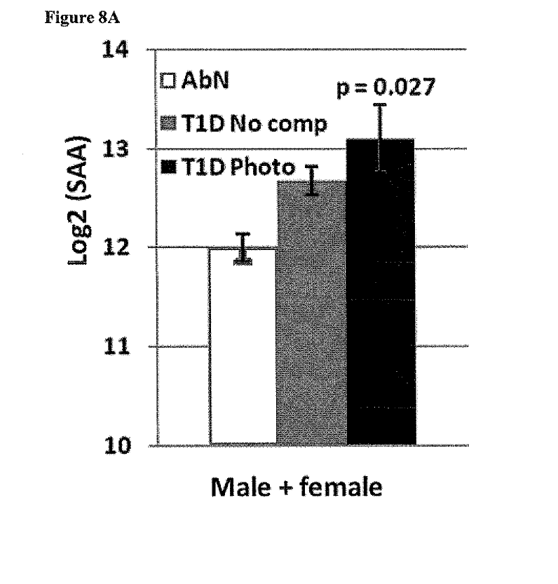

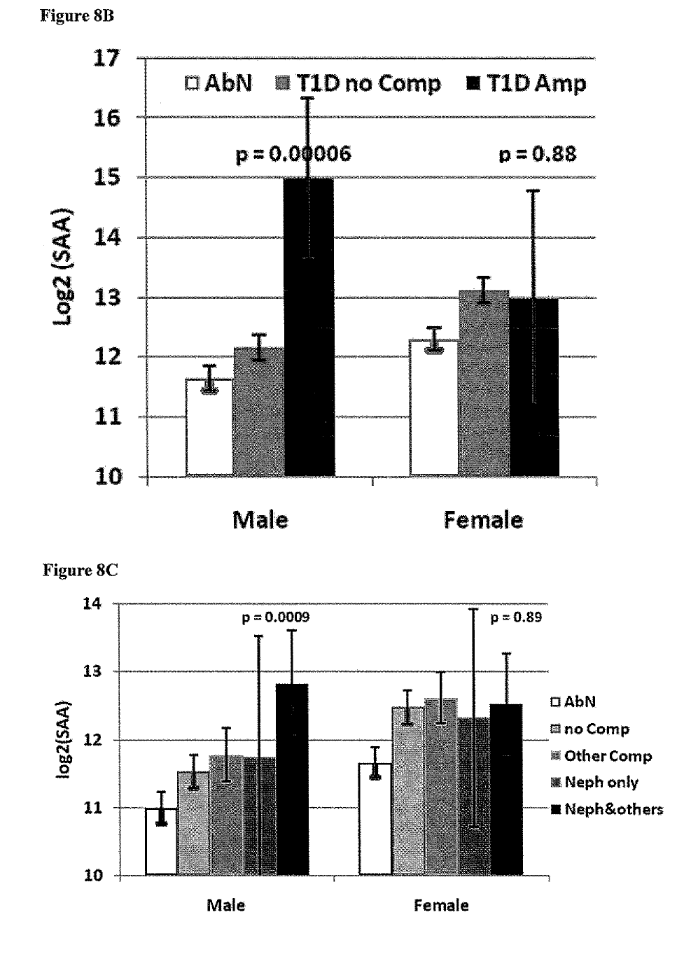

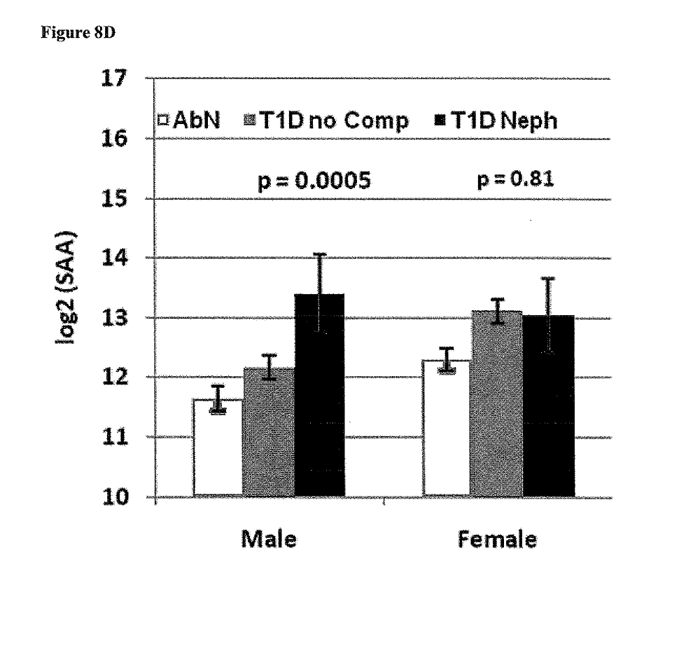

The association between SAA levels and T1D complications was examined using regression analyses that included four phenotypic groups (AbN, T1D patients without any complication, T1D patients with a complication other than the one being examined, and T1D patients with each specific complication), age and gender. These analyses indicated significant differences in SAA levels among the four phenotypic groups and significant interaction with gender for nephropathy and amputation. Subsequently, potential differences were examined between T1D patients without any diabetic complication (n=611) and healthy controls or T1D patients with each complication. The analysis revealed significantly higher SAA levels in T1D patients without any complication compared to healthy controls (p=2.5×10⁻⁸). Interestingly, patients with any complication did not differ significantly from patients without any complication. However, SAA levels for T1D patients with photocoagulation were marginally significantly higher than those for patients without any diabetic complication (p=0.027, FIG. 8A). The analyses for amputation and nephropathy revealed a significant interaction with gender. These analyses revealed significantly higher SAA levels in male T1D patients with amputation compared to male T1D patients without any complication (p=6×10⁻⁵, FIG. 8B), but no difference in females (p=0.88). Similarly, male T1D patients with nephropathy had significantly higher SAA levels compared to male patients without any complication (p=0.0005, FIG. 8C), again with females showing no difference (p=0.82). Further analyses suggested that patients with nephropathy alone did not have significantly higher SAA but patients with nephropathy and other complications had higher SAA compared to patients without any complication (FIG. 8D). Finally, the relationship was examined between SAA levels and the number of complications but did not identify a significant relationship probably due to small sample sizes after dividing the subjects into multiple phenotypic groups.

### Example 6

### Changes in Serum Protein Levels in Diabetic Patients with Complications

The association between IGFBP2 (FIG. 9A), IL1RA (FIG. 9B), IL8 (FIG. 9C), MCP1 (FIG. 9D) and MIP1B (FIG. 9E) levels and T1D complications was examined using regression analyses that included four phenotypic groups (AbN (n=673), T1D patients without any complication (n=401), T1D patients with a complication other than the one being examined (n=183-288), and T1D patients with each specific complication (n=10-113)), age and gender. These analyses indicated significant differences in level of 5 proteins among the four phenotypic groups. Protein MIP1b was significantly associated with retinopathy, nephropathy, cardiovascular disease and hypertension. Significant interaction with gender was observed for nephropathy and blindness for protein MCP1. Subsequently, potential differences were examined between T1D patients without any diabetic complication (n=401) and healthy controls or T1D patients with each complication. Interestingly, patients with complication differ significantly from patients without any complication for IGBFP2, IL1RA, IL8, MCP1, MIP1B.

The analysis revealed significantly higher IGFBP2 levels in T1D patients without any complication compared to healthy controls (p=5×10⁻³). Elevated levels of IGFBP2 were observed for T1D patients with nephropathy, blindness, cardiovascular disease and hypertension. T1D patients with these complications had 3-10 fold higher levels of IGBFP2 levels than those without any diabetic complication (p=1×10⁻³−4×10⁻¹³, FIG. 9A,10A,11A,12A). Regression analyses for the complications revealed a no significant interaction with gender and the serum levels of IGFBP2 was similar for both males and females with or without any complications (p=0.066).

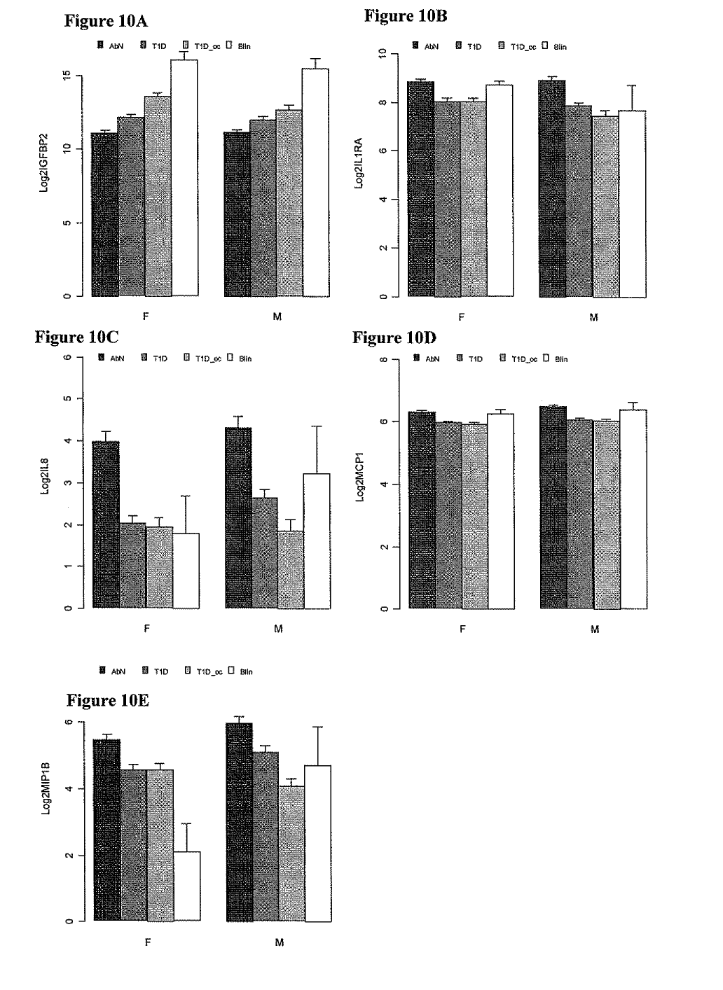

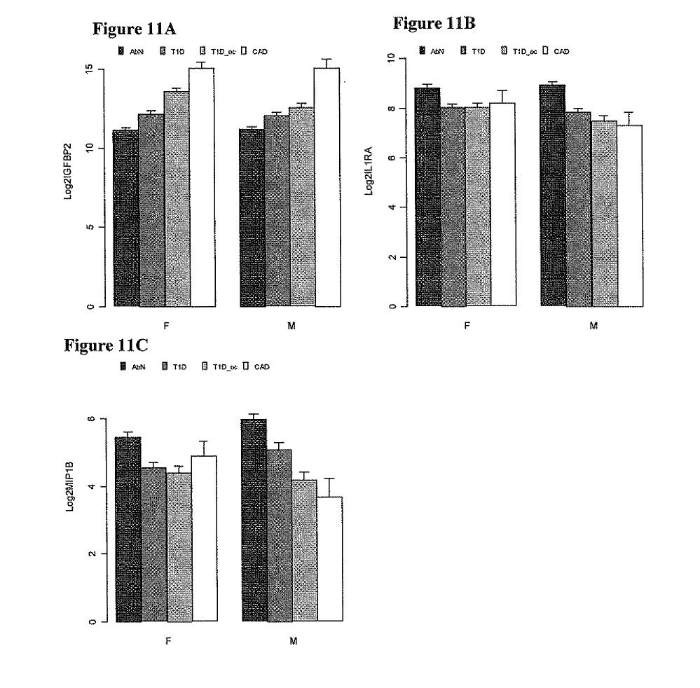

Healthy controls have elevated levels of IL1RA compared to the T1D subjects without any complications (FIG. 9B, 10B, 11B). Female T1D subjects with nephropathy have similar levels of IL1RA, marginally increased levels of IL1RA (FIG. 9B) was observed for male T1D patients with nephropathy than female T1D patients without nephropathy (p=0.0069). Regression analyses for the nephropathy revealed a no significant interaction with gender and the serum levels of IL1RA. Marginal elevation in serum levels of IL1RA was observed in female T1D patients with blindness compared to female T1D subjects without any complications (p=0.09, FIG. 10B). However no significant differences were observed for male T1D patients with or without blindness (FIG. 10B). Reduction in serum levels of IL1RA was noted for male T1D patients with cardiovascular disease compared to male T1D patients without cardiovascular disease (FIG. 11B, p=0.003)

Reduction in serum levels of MCP1 were found between healthy controls and T1D patients without any complications (FIGS. 9D and 10D, p=1×10⁻¹⁰). MCP1 levels were similar for T1D females with or without any nephropathy (p=0.22). However elevated levels of MCP1 were found in male T1D patients with nephropathy compared to male T1D patients without nephropathy (FIG. 9D, p=0.006). T1D patients with blindness had marginally elevated levels of IL-1RA compare to T1D patient without blindness or with other complications (FIG. 10D, p=0.006). No differences in MCP levels were observed between T1D patients and T1D patients with other complications for both male and female T1D patients when analysis was done for blindness group (FIG. 10D).

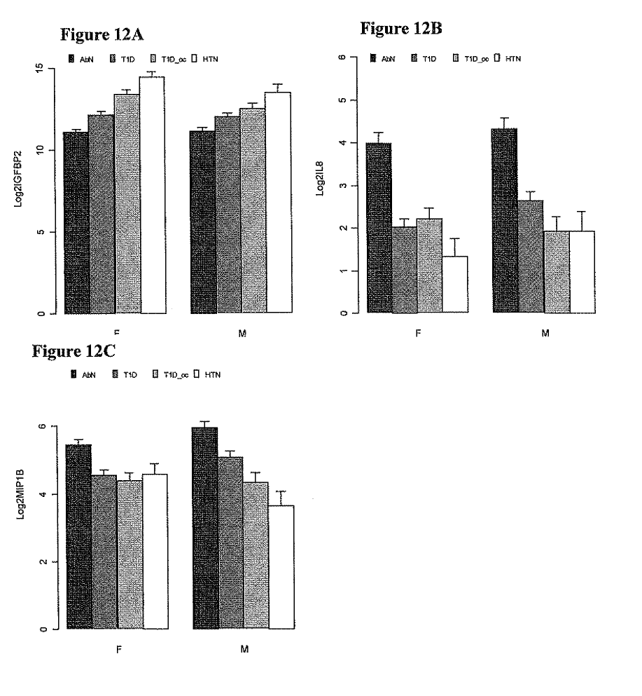

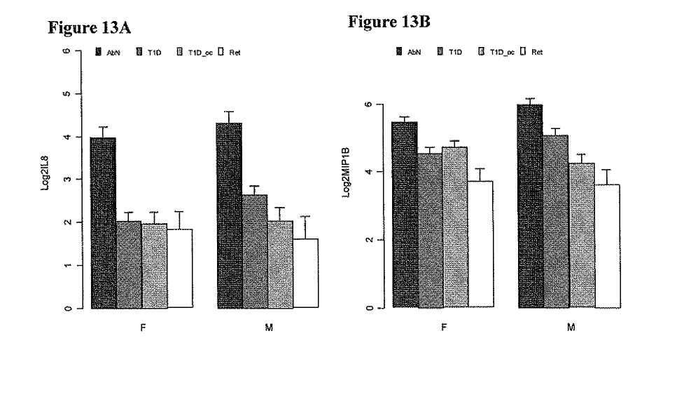

IL8 was reduced in T1D patients without any complications in comparison to healthy controls (FIGS. 9C, 10C, 12B and 13A, p=1.92×10⁻²⁸). Analyses revealed marginally higher IL8 (FIG. 9C) levels in male T1D patients than female T1D subjects (FIGS. 9C, 10C, 12B and 13A, p=2×10⁻³). No significant differences were noted for female T1D patients with other complications compared to female T1D patients (FIG. 9C, 10C, 12B, 13A). Male T1D patients with nephropathy (p=6×10⁻⁶, FIG. 9C) and retinopathy (FIG. 13A) had lowered levels of IL8 compared to female T1D patients without nephropathy and retinopathy. Male T1D subjects with blindness (FIG. 10C), hypertension (FIG. 12B) had a higher levels of IL8 compared to female T1D patients with blindness and hypertenstion. In hypertensive T1D patients IL8 was lowered significantly compared to T1D patients without hypertension (FIG. 12B, p=7.7×10⁻⁸)

Analysis of MIP1B levels revealed to be increased healthy controls compared to T1D patients (FIGS. 9E, 10E, 11C, 12C and 13B). MIP1B was elevated in female T1D patients with nephropathy (FIG. 9E, p=0.003) compared to female T1D patients, whereas no significant differences were found for male phenotypic group for nephropathy. A reduction in mean serum levels of MIP1B was observed in female T1D patients with blindness compared to female T1D patients, with males showing no significant differences between the two phenotypic groups. Male T1D patients with blindness showed elevated levels of MIP1B compared to female T1D patients with blindness (FIG. 10E, p=0.01). MIP1B was reduced in male T1D patients with cardiovascular disease (FIG. 11C, p=0.004) and hypertension (FIG. 12C, p=0.0001) when compared to female T1D patients with and without cardiovascular disease and hypertension. Reduced levels of MIP1B were associated with T1D patients with retinopathy, irrespective of their gender (FIG. 13B, p=0.005 and 5×10-5)

We claim:

1. A method of determining a subject's risk of developing type 1 diabetes (T1D) complications, comprising measuring in a sample from the subject the levels of one or more serum proteins selected from the group consisting of interleukin-1-receptor antagonist (IL-1ra), monocyte inhibitory protein 1 beta (MIP-1β), Interleukin 8 beta (IL-8), monocyte chemotactic protein 1 beta (MCP-1), and myeloperoxidase (MPO), wherein detection of low levels of serum proteins compared to a control is an indication that the subject is at risk of developing T1D.

2. The method of claim 1, wherein the low levels of the serum proteins compared to a control is an indication that the subject is at risk of developing islet autoimmunity.

3. The method of claim 1, wherein the control is a T1D patient.

4. The method of claim 2, wherein detection of less than about 1024 pg/ml IL-1ra in the sample is an indication that the subject is at risk of developing islet autoimmunity and T1D.

5. The method of claim 2, further comprising assaying the sample for the presence of autoantibodies against islet antigens,
wherein detection in the sample of low levels of serum proteins compared to a control but not the presence of autoantibodies against islet antigens is an indication that the subject is at risk of developing islet autoimmunity, and
wherein detection in the sample of autoantibodies against islet antigens and low levels of serum proteins compared to a control is an indication that the subject has islet autoimmunity and is at risk of developing T1D.

6. A method of determining a subject's risk of developing type 1 diabetes (T1D), comprising measuring in a sample from the subject the levels of serum proteins selected from the group consisting of serum amyloid A (SAA), insulin growth factor binding protein 2 (IGFBP2), and Adiponectin, wherein detection of elevated levels of the serum proteins compared to a control is an indication that the subject is at risk of developing T1D.

7. The method of claim 6, wherein elevated levels of the serum proteins compared to a control is an indication that the subject is at risk of developing islet autoimmunity.

8. The method of claim 7, further comprising assaying the sample for the presence of autoantibodies against islet antigens,
wherein detection in the sample of elevated levels of the serum proteins but not the presence of autoantibodies against islet antigens is an indication that the subject is at risk of developing islet autoimmunity, and
wherein detection in the sample of autoantibodies against islet antigens and elevated levels of the serum proteins is an indication that the subject has islet autoimmunity and is at risk of developing T1D.

9. The method of claim 1 or 6, wherein the sample is blood, plasma, or serum isolated from the subject.

10. The method of claim 1, wherein the subject has one or more T1D susceptibility genes.

11. A method of treating or preventing islet immunity in a subject, comprising administering to the subject a composition comprising an interleukin-1-receptor antagonist (IL-1ra) agonist, a myeloperoxidase (MPO) agonist, monocyte inhibitory protein 1 beta (MIP-1β) agonist, Interleukin 8 beta (IL-8) agonist, monocyte chemotactic protein 1 beta (MCP-1) agonist, or a combination thereof.

12. The method of claim 11, wherein the IL-1ra agonist is recombinant IL-1ra.

13. The method of claim 11, wherein the method comprises measuring the levels of IL-1ra in a sample from the subject, wherein the IL-1ra agonist is administered to subjects wherein the sample from the subject has less than about 200 pg/ml IL-1ra.

14. The method of claim 11, wherein the MPO agonist, MIP-1β agonist, IL-8 agonist, or MCP agonist is a recombinant protein.

15. The method of claim 11, wherein the method comprises measuring the levels of MPO, MIP-1β, IL-8, or MCP in a sample from the subject, wherein the MPO agonist, MIP-1β agonist, IL-8 agonist, MCP agonist, or combination thereof is administered to subjects having low levels of MPO, MIP-1β, IL-8, or MCP compared to a control.

16. A method of treating or preventing islet immunity in a subject, comprising administering to the subject a composition comprising a serum amyloid A (SAA) antagonist, a IGFBP2 antagonist, an Adiponectin antagonist, or a combination thereof.

17. The method of claim 16, wherein the SAA antagonist, IGFBP2 antagonist, or Adiponectin antagonist is a therapeutic antibody that selectively binds SAA, IGFBP2, or Adiponectin and inhibits its activity.

18. The method of claim 16, wherein the SAA, IGFBP2 or Adiponectin antagonist is a therapeutic small molecule that binds SAA, IGFBP2, or Adiponectin and inhibits its activity.

19. The method of claim 16, wherein the method comprises measuring the levels of SAA, IGFBP2 or Adiponectin in a sample from the subject, wherein the SAA antagonist, IGFBP2 antagonist, Adiponectin antagonist, or combination thereof is administered to subjects having elevated levels of SAA, IGFBP2, or Adiponectin compared to a control.

20. The method of claim 11, wherein the sample is blood, plasma, or serum isolated from the subject.
